(19) United States
(12) Patent Application Publication
(10) Pub. No.: US 2026/0290505 A1
(43) Pub. Date: Sep. 24, 2026

(54) A METHOD FOR PREDICTING ONE OR MORE SAMPLE METRIC VALUES BY MACHINE LEARNING

(71) Applicant: Thermo Fisher Scientific (Bremen) GmbH, Bremen (DE)

(72) Inventor: Klaus Dennis Linke, Hellwege (DE)

(73) Assignee: Thermo Fisher Scientific (Bremen) GmbH, Bremen (DE)

(21) Appl. No.: 19/479,210

(22) PCT Filed: Apr. 16, 2024
(86) PCT No.: PCT/EP2024/060316

§ 371 Date: Oct. 27, 2025

(30) Foreign Application Priority Data
Apr. 28, 2023 (GB) 2306359.7

(51) Int. Cl. G16B 40/00; G01N 30/02; G01N 30/72; G01N 30/86; G01N 30/88

(52) U.S. Cl. CPC G16B 40/00; G01N 30/7233; G01N 30/8693; G01N 30/8696; G01N 30/88; G01N 2030/027; G01N 2030/8831

(57) ABSTRACT

A method for predicting one or more sample metric values by machine learning is provided. The method comprises determining a plurality of characteristics from raw data obtained by analysing a proteomics sample in a liquid chromatography mass spectrometer, LC-MS. The method further comprises predicting one or more sample metric values by processing the plurality of characteristics using one or more trained machine learning models.

### FIELD OF THE INVENTION

The invention relates to a new method for predicting sample metrics by a data reduction step in combination with machine learning. Specifically, the invention relates to reducing the processing time by utilizing only a representative subset of information contained in Mass Spectrometry raw files. The invention may be used during quality control (QC) to access the performance of liquid chromatography mass spectrometry (LC-MS) instruments.

The invention may also be used to estimate the quality of the raw file generated by liquid chromatography mass spectrometry (LC-MS) instruments.

### BACKGROUND

In mass spectrometry, a sample is ionised and the resulting ions categorised according to their mass to charge ratio (m/z). The output of a typical mass spectrometer is a spectrum showing the distribution of m/z detected by the system, which must be carefully interpreted to provide information about the sample.

FIG. 1 shows a schematic diagram of a mass spectrometer that can be used to carry out the invention. Such a mass spectrometer is described in detail in WO2012/160001. The mass spectrometer 2 is shown in which ions are generated from a sample in an ion source (not shown), which may be a conventional ion source such as an electrospray. Ions may be generated as a continuous stream in the ion source as in electrospray, or in a pulsed manner as in a Matrix Assisted Laser Desorption/Ionization “MALDI” source. The sample which is ionised in the ion source may come from an interfaced instrument such as a liquid chromatograph (not shown). The ions pass through a heated capillary 4, are transferred by an RF only S-lens 6, and pass the S-lens exit lens 8. The ions in the ion beam are next transmitted through an injection flatapole 10 and a bent flatapole 12 which are RF only devices to transmit the ions, the RF amplitude being set mass dependent. The ions then pass through a pair of lenses and enter a mass resolving quadrupole 18.

The differential RF and DC voltages of the quadrupole 18 are controlled to either transmit all ions (RF only mode) or select ions of a particular m/z range for transmission by applying RF and DC according to the Mathieu stability diagram. It will be appreciated that, in other embodiments, instead of the mass resolving quadrupole 18, an RF only quadrupole or multipole may be used as an ion guide but the spectrometer would lack the capability of mass selection before analysis. In still other embodiments, an alternative mass resolving device may be employed instead of quadrupole 18, such as a linear ion trap, magnetic sector or a time-of-flight analyser. Such a mass resolving device could be used for mass selection and/or ion fragmentation. Turning back to the shown arrangement, the ion beam which is transmitted through quadrupole 18 exits from the quadrupole through a quadrupole exit lens 20 and is switched on and off by a split lens 22. Then the ions are transferred through a transfer multipole 24 (RF only, RF amplitude may be set mass dependent) and collected in a curved linear ion trap (C-trap) 26. The ions are trapped radially in the C-trap by applying RF voltage to the curved rods of the trap in a known manner. The C-trap is elongated in an axial direction (thereby defining a trap axis) in which the ions enter the trap. Voltage on the C-Trap exit lens 28 can be set in such a way that ions cannot pass and thereby get stored within the C-trap 26. Similarly, after the desired ion fill time (or number of ion pulses e.g. with MALDI) into the C-trap has been reached, the voltage on C-trap entrance lens 30 is set such that ions cannot pass out of the trap and ions are no longer injected into the C-trap. More accurate gating of the incoming ion beam is provided by the split lens 22.

Ions which are stored within the C-trap 26 can be ejected orthogonally to the axis of the trap (orthogonal ejection) by pulsing DC to the C-trap in order for the ions to be injected, in this case via Z-lens 32, and deflector 33 into a mass analyser 34, which in this case is an electrostatic orbital trap, and more specifically an Orbitrap™ FT mass analyser made by Thermo Fisher Scientific. The orbital trap 34 comprises an inner electrode 40 elongated along the orbital trap axis and a split pair of outer electrodes 42, 44 which surround the inner electrode 40 and define therebetween a trapping volume in which ions are trapped and oscillate by orbiting around the inner electrode 40 to which is applied a trapping voltage whilst oscillating back and forth along the axis of the trap. The pair of outer electrodes 42, 44 function as detection electrodes to detect an image current induced by the oscillation of the ions in the trapping volume and thereby provide a detected signal. The outer electrodes 42, 44 thus constitute a first detector of the system. The outer electrodes 42, 44 typically function as differential pair of detection electrodes and are coupled to respective inputs of a differential amplifier (not shown), which in turn forms part of a digital data acquisition system (not shown) to receive the detected signal. The detected signal can be processed using Fourier transformation to obtain a mass spectrum.

The mass spectrometer 2 further comprises a collision or reaction cell 50 downstream of the C-trap 26. Ions collected in the C-trap 26 can be ejected orthogonally as a pulse to the mass analyser 34 without entering the collision or reaction cell 52 or the ions can be transmitted axially to the collision or reaction cell for processing before returning the processed ions to the C-trap for subsequent orthogonal ejection to the mass analyser. The C-trap exit lens 28 in that case is set to allow ions to enter the collision or reaction cell 50 and ions can be injected into the collision or reaction cell by an appropriate voltage gradient between the C-trap and the collision or reaction cell (e.g. the collision or reaction cell may be offset to negative potential for positive ions). The collision energy can be controlled by this voltage gradient. The collision or reaction cell 50 comprises a multipole 52 to contain the ions. The collision or reaction cell 50, for example, may be pressurised with a collision gas so as to enable fragmentation (collision induced dissociation) of ions therein, or may contain a source of reactive ions for electron transfer dissociation (ETD) of ions therein. The ions are prevented from leaving the collision or reaction cell 50 axially by setting an appropriate voltage to a collision cell exit lens 54. The C-trap exit lens 28 at the other end of the collision or reaction cell 50 also acts as an entrance lens to the collision or reaction cell 50 and can be set to prevent ions leaving whilst they undergo processing in the collision or reaction cell if need be. In other embodiments, the collision or reaction cell 50 may have its own separate entrance lens. After processing in the collision or reaction cell 50 the potential of the cell 50 may be offset so as to eject ions back into the C-trap (the C-trap exit lens 28 being set to allow the return of the ions to the C-trap) for storage, for example the voltage offset of the cell 50 may be lifted to eject positive ions back to the C-trap. The ions thus stored in the C-trap may then be injected into the mass analyser 34 as described before.

The mass spectrometer 2 optionally further comprises an electrometer 60 which is situated downstream of the collision or reaction cell 50 and can be reached by the ion beam through an aperture 62 in the collisional cell exit lens 54. The electrometer 60 may be either a collector plate or Faraday cup and is connected to a high gain charge sensitive amplifier. It will be appreciated, however, that the electrometer 60 in other arrangements may be another type of charge measuring device. Preferably, the electrometer is of differential type which reduces noise pick-up from other electrical sources nearby. A first input of the electrometer is arranged to receive current or charge from the ion source while another input is arranged to have similar capacitance, dimensions and orientation to the first input but receives no ion current or charge at all. The electrometer 60 thus constitutes an optional second detector of the system, which is independent of the first detector, namely the image current detection electrodes 42, 44 of the mass analyser 34. In some arrangements the collision or reaction cell 50 may not be present, in which case the electrometer 60 is preferably located downstream of the C-trap behind C-trap exit lens 28.

It will be appreciated that the path of the ion beam through the spectrometer and in the mass analyser is under appropriate evacuated conditions as known in the art, with different levels of vacuum appropriate for different parts of the spectrometer.

It is to be understood, that any other mass spectrometer may be as well suited for use in connection with this invention. The Orbitrap mass analyser could e.g. be replaced by a time of flight analyser, or mass spectrometer 2 could be a triple quadrupole mass spectrometer, an ion trap mass spectrometer, or a quadrupole- or ion trapping quadrupole-time of flight mass spectrometer. The mass spectrometer could comprise more than one mass analyser; for example the mass spectrometer could be the hybrid Orbitrap™ mass analyser and time of flight mass analyser instrument described in EP3410463.

The mass spectrometer 2 is under the control of a control unit, such as an appropriately programmed computer (not shown), which controls the operation of various components and, for example, sets the voltages to be applied to the various components and which receives and processes data from various components including the detector(s). The computer is configured to use a known algorithm to determine the settings (e.g. injection time or number of ion pulses) for the injection of ions into the C-trap for analytical scans in order to achieve the desired ion content (i.e. number of ions) therein which avoids space charge effects whilst optimising the statistics of the collected data from the analytical scan. The algorithm may rely on preceding measurements of the mass analyser 34 or electrometer 60.

Mass spectrometry is particularly useful for the identification of proteins (or, in general, other compounds hereafter referred to as “proteins”) within an organic sample, a field called proteomics, which is an important aspect of biological and medical research.

In one method of analysing organic samples, sometimes known as “bottom-up” mass spectrometry, proteins are pre-digested into their constituent peptides (or, in general, any components of a compound, hereafter referred to as “peptides”), which are examined and classified in a mass spectrometer. Several databases exist that are able to provide the spectra expected for any given peptide. Therefore, if only a small number of proteins are present in the original sample, it is relatively straightforward to compare the spectrum of the peptides identified in the mass spectrometer with known spectra from various predetermined proteins and to identify the closest match, and so identify the most likely protein that was present in the sample.

The development of large protein databases has made it possible to identify many otherwise unidentified proteins by comparing information from their analysis, such as their sequences or mass spectra, with information in or from the database. Developments in high-throughput peptide analysis techniques, such as robotic gel band excision and digestion, and matrix-assisted laser desorption/ionization (MALDI) mass spectrometry, have made it possible to collect large volumes of data that characterise large numbers of experimental proteins. Such information can be compared with information in databases of known proteins in order to identify such experimental proteins.

Mass spectrometry (MS) is particularly well suited to the analysis of these peptides, especially when used in conjunction with liquid chromatography (LC). With the use of LC/MS, the peptides of proteins that have been proteolytically digested are separated using methods of LC. A mass spectrometer then analyses the peptides according to their relative mass-to-charge ratio (m/z), producing a characteristic spectrum of peaks for the peptide, which may belong to one or more proteins. With the use of tandem mass spectrometry (MS/MS), a single peptide of a protein can be selected and subjected to collision-induced dissociation (CID) or another fragmentation technique. CID produces fragment ions that may then be sorted according to their mass-to-charge ratios, producing a characteristic spectrum for the selected peptide. The repeated application of liquid chromatography tandem mass spectrometry (LC-MS/MS) can produce a large number of spectra, each characterizing a plurality of different peptides.

A protein that has been characterized by methods such as LC-MS/MS can be identified by comparing its experimental data such as the mass spectra of its peptides with characteristic data such as theoretical mass spectra for peptides of previously identified (“known”) proteins. By comparing the experimental data of an unknown peptide to theoretically derived properties of known peptide sequences, the unknown peptide as well as the unknown protein to which the unknown peptide belongs can be identified.

Searchable protein databases are available, e.g., at the National Center for Biotechnology Information (NCBI) website (http://www.ncbi.nlm.nih.gov). They include databases of nucleotide sequence information and amino acid sequence information for proteins.

To evaluate MS/MS data for peptides using a nucleotide or protein sequence database, sequences in the database that represent proteins can be divided into sequences representing the peptides that would result from an actual proteolytic digestion of the proteins. A theoretical spectrum can then be generated for each peptide of a protein represented in the database, based on the sequence of the peptide. The theoretical spectrum includes mass-to-charge peaks that would be expected if the protein in the database were subjected to MS/MS and the peptide of interest was selected for characterization. Each theoretical peptide spectrum for proteins represented in the database can be compared to observed peptide spectra for an unknown protein. The similarity of the theoretical peptide spectra to the unknown peptide spectra can then be used to determine the identity of the unknown protein. The SEQUEST or MASCOT search engines implement such a routine for protein identification. See, for example, Eng J K, McCormack A L, and Yates J R 3rd, “An Approach to Correlate Tandem Mass Spectral Data of Peptides with Amino Acid Sequences in a Protein Database”, J. Am. Soc. Mass. Spectrom., 1994, 5:976-989.

Modern software applied in Proteomics performs many different steps to analyse raw files. Generally, these steps can include (and not limited to): (i) read in the raw file to get MS and MS/MS information, (ii) extract the relevant information from the MS/MS spectrum (iii) conduct a database search as outlined above (SEQUST), (iv) extract information about quantification [if applied], (v) perform false discovery identification to differentiate between true and false positive hits (vi) report sample metrics such as ‘Number of Proteins’, ‘Number of Protein Groups’, ‘Number of Peptide Groups’, ‘Number of Peptide Spectrum Matches’.

These sample metrics can be described as:
Number of Peptide Spectrum Matches (PSMs):
   Is the number of MS/MS spectra that were matched to peptide sequences for a given protein.
Success Rate
   Is calculated by dividing the Number of Peptide Spectrum Matches (PSMs) by the Number of MS/MS scans acquired.
Number of Peptide Groups
   Is the number of Peptide Sequences obtained after grouping Peptide Spectrum Matches based on their sequence and modification.
Number of Proteins:
   Represents the number of proteins identified.
Number of Protein Groups:
   A reduced number of Proteins. A protein group is a set of proteins, which cannot be unambiguously identified by unique peptides. These are grouped into a protein group.

Processing proteomic data as indicated above can be a time-consuming and computationally intensive step. This is especially true for modern search engines such as CHIMERYS. Resources (time, money and/or electrical energy) should ideally only be invested for already acquired raw files which are expected to provide useful sample metrics (such as Number of Protein Groups). Additionally, new raw files should only be acquired if the LC-MS setup operates in an expected performance. The latter approach is often ensured by running Quality Control (QC) samples. QC runs are used to validate LC-MS application system performance. Here, QC runs are acquired using standardized methods, which are as close as possible to the LC-MS settings applied to collect data from real/biological samples. These runs therefore also use a sample resembling the real biological samples. Data obtained during the QC run may be used to determine whether the spectrometer is operating correctly. If not, maintenance operations may be carried out on the spectrometer to restore performance. QC runs may be performed as close as possible to the runs during which the real samples are analysed. These QC-runs can therefore be acquired immediately before, immediately after or interspersed between running biological/real samples. To ensure validity of the QC runs and reduce the overall time taken for the LC-MS sequence, it is important to process and evaluate the data files from the QC runs as quickly as possible to mitigate the ‘pending-time’ of an LC-MS setup while waiting for the outcome of the QC run.

Three approaches are currently applied to reduce the ‘pending-time’ of an LC-MS setup while analysing raw files for QC purposes or to estimate if the quality of a biological sample is high enough to invest additional time to process this data:

### a) Run Only a Portion of the Database Search Workflow.

In this case, mostly, the quantification part of the database search is omitted to decrease the overall search time. One drawback of this approach is that the database search is limited to identification (e.g., protein groups). If quantification results are also needed (e.g., to estimate the quality of a QC run additionally), the database search needs to be repeated to get results for the identification and quantification. This can block additional hardware resources to process other raw files.

b) Run the QC Files with a Different Search Engine.

By using a search engine/algorithm which is designed for speed, the required time for processing can be reduced significantly. The drawbacks of this approach include (i) increasing the amount of processed result files, (ii) the need to trigger this tool via an external process and (iii) to map the results from the quick search to the results from the original workflow.

### c) in Another Approach, the File Size of Raw Files Produced by the Mass Spectrometer (MS) is Used as a Very Approximate QC Metric.

The current methods for processing proteomics data suffer from the drawbacks described above. A new method that is fast, lightweight, and reliable is therefore desired.

### SUMMARY

The invention seeks to provide an improved method for reducing the time required to process data from proteomics runs (such as QC-runs) to predict commonly applied sample metrics (e.g., ‘Number of Protein Groups’) by machine learning, to estimate its data quality. The proposed methods may be used for quickly analysing proteomics raw files acquired with a standardized LC-MS method, independently of a database search approach. Based on the outcome, the data can be further processed with modern post-acquisition software applied in proteomics.

One or more sample related metrics may be obtained in prior art methods by processing raw files produced by the LC-MS using a bioinformatics software such as the “Proteome Discoverer” software from Thermo Fisher Scientific.

The one or more sample metrics for proteomics (and proteomics QC-) may comprise:
Number of Proteins;
Number of Protein Groups;
Number of Peptide Groups;
Number of Peptide-Spectrum-Matches; and/or
Success Rate (=Number of Peptide-Spectrum-Matches/Number of MS/MS Scans acquired).

However, running bioinformatics software suites requires considerable computing resources and time. Therefore, instead of processing the data files acquired using standardized LC-MS methods with computer-intensive database searches (or a search engine designed for speed, as in other prior art methods), the proposed methods extract representative data from the raw data files. Machine learning approaches are then used to predict one or more sample metrics.

By doing so, the time taken to estimate the one or more sample metrics (see list above) may be reduced, compared to prior art methods.

A method which includes data extraction from the raw file(s), reduction of the data complexity and training a machine learning model for quickly predicting sample metrics to avoid time-consuming and complex calculations in proteomics applications is therefore desired.

According to the present invention, a method for predicting one or more sample metric values by machine learning is provided. The method comprises:
determining a plurality of characteristics from raw data obtained by analysing a proteomics sample in a liquid chromatography mass spectrometer, LC-MS; and
predicting one or more sample metric values by processing the plurality of characteristics determined from the raw file using one or more trained machine learning models.

The plurality of characteristics may be representative of the performance of the LC-MS. In other words, the characteristics may be indicative of a quality of the raw data obtained.

Since the raw data is obtained by analysing a proteomics sample in a liquid chromatography mass spectrometer, LC-MS, the raw data may be referred to as LC-MS raw data.

The proteomics sample may be a standardized proteomics sample. In other words, the sample may be of a known composition. Analysis of standardized proteomics samples is useful for validating instrument performance, for calibrating instrument parameters and for training data analysis methods, such as machine learning models.

In one example, the proteomics sample may be a quality control (QC) sample. In this case, the one or more sample metric values may comprise one or more QC metrics. The one or more QC metrics may be used to determine a QC outcome during a QC run, to validate instrument performance.

One important contribution over the prior art is that the raw data is manipulated to reduce the complexity (and data size), while retaining useful characterising information that enables the prediction methods to provide reliable outcomes. Manipulation of the raw data (extraction of the representative characteristics from the raw data), in combination with a machine learning algorithm to process these characteristics and predict one or more sample metric values such as the number of protein groups, is not taught in any of the prior art.

The sample metric to be predicted by a data reduction step followed by machine learning may be a number of protein groups in the proteomics sample identifiable by the LC-MS. Alternatively, the sample metric may be a number of peptide groups in the proteomics sample identifiable by the LC-MS. Alternatively, the sample metric may be a number of peptide spectrum matches (PSMs) in the proteomics sample identifiable by the LC-MS. Alternatively, the sample metric may be a number of peptide spectrum matches divided by the Number of MS/MS spectra acquired in the proteomics sample identifiable by the LC-MS (defined as success rate=PSMs/Number of MS/MS Spectra)

The number of protein groups is a sample metric that is applied in prior art proteomics methods to assess the quality of the raw files, especially when analysing QC runs. The value of this metric may be related to a combination of other parameters (including the number of peptide groups and peptide spectrum matches) and filter options. Therefore, the value of this metric is relatively robust in a controlled environment (i.e., using the same standardized sample, and the same standardized LC-MS method). Although the value of this sample metric is a combination of several features, a decrease in the number of protein groups is an indicator that the LC-MS system (including samples and solvents) is not performing as expected. In contrast to the number of protein groups, the number of Peptide Spectrum Matches (i.e., how many MS/MS spectra could be mapped to a peptide sequence) is not based on any other sample metric described above (i.e., Peptide Groups, Proteins or Protein Groups) but is directly linked to the quality of the MS/MS spectra.

The quality of the MS/MS spectra collected are highly and directly dependent on the performance of the LC-MS system (including the solvents, sample, column, LC and MS; and the LC-MS method applied). As a particular Peptide Spectrum Match (PSM) is linked to a particular MS/MS event during elution of the sample, this PSM (and its successor Success Rate) provides an (identification based) sample metric which can be visualized effectively in a LC-MS runtime plot (i.e., Number of Peptide Spectrum Matches (y-axis) vs the Retention Time (x-axis)). This plot can be used to evaluate visually if two QC runs show different characteristics based on the number of PSMs obtained at a given retention time. One such characteristic difference could be a shift to higher retention times.

In some examples, the sample metric values (such as Number of Proteins, Number of Protein Groups, Number of Peptide Groups, Number of Peptide-Spectrum-Matches, or Success Rate) may also be calculated by Proteome Discoverer. In some cases, the sample metric values are related. For example, Peptide-Spectrum-Matches (“PSMs”) may be combined when specific features are matching to build a Peptide Group. Multiple Peptide Groups can form a Protein Group, when specific features are matching. In other words, a Number of Protein Groups may be the downstream product of PSMs and Peptide Groups. In some cases, the Number of Peptide Groups and PSMs may be affected by smaller changes (LC, MS, Sample, . . . ) than the other metrics. Therefore, the Number of Protein Groups may be a preferred sample metric.

Other characteristics to assess the quality of a raw file might include the number of unfragmented (MS1) and fragmented (MS2) spectra. An MS spectrum can be composed of many peaks with different intensities (hundreds of peaks per scan, in some cases). The complexity of the raw data may be reduced prior to processing (in some cases, to a “least necessary minimum”). In some examples, this data reduction may be achieved by reporting a median value, an absolute value, or a relative value. All these features used for ‘data reduction’ are extracted from the raw file (e.g., by exporting values from the scan header or from the MS spectrum directly). An example for data extracted from the scan header is the Total Ion Current (TIC). Information extracted from MS or MS/MS spectra might be the total number of peaks per spectrum or the median intensity of peaks within a particular spectrum.

Information extracted from the raw file may further be grouped into the MS-n level. Commonly, this level ranges from 1 to 2 (i.e., MS1 and MS2) for identification based routine analysis by LC-MS in proteomics experiments. Further grouping may be based on other specific feature(s). An example could be to sort the data further into sub-groups if the ion injection time was below or at the maximal allowed ion injection time, respectively, while acquiring an MS or MS/MS spectrum.

This is different to commonly applied methods to obtain sample metric values (such as the number of protein groups) where information from (ideally) each MS or MS/MS spectrum (i.e., m/z and its intensity pair) is used as input features. In the approach described above, only one value for one feature is used as a representative value to describe a raw file.

Additional information about the different data reduction steps and examples are provided below:

### 1. Median

For example, the total ion current (TIC) MS1, which changes through the proteomics run depending on the retention time when peptides (and other substances) are detected by the Mass Spectrometer. The median value of all MS1 TIC values should only differ to a very small portion between different raw files acquired using the same sample and LC-MS method.

### 2. Absolute Value

For example, the number of MS1 scans, which is an aggregated value. This is reported as the “total number of MS1 scans”. This value is also very constant when the LC-MS setup is kept constant and is not negatively affected.

### 3. Relative Value

For example, the ratio of Number of MS1 Scans to Number of MS2 Scans (MS1/MS2), which may provide another useful characteristic of the raw data.

The complexity of the data contained in the raw file may be reduced by extracting these characteristic features directly from the raw file (e.g., from the raw file scan header) while eliminating the retention time domain from the processed data. This reduced data set may be paired with outcomes from a database search engine, to train a machine learning model. This trained machine learning model may then be used to predict the sample metric (e.g., Number of Protein Groups) using data which the machine learning model has not seen before (‘new data’). The new data may be extracted from a new raw file that was not used for training the ML model. To predict the sample metric value(s) (e.g., number of protein groups) quickly, the new data provided to the trained model may comprise features extracted from the new raw file, instead of providing the entire new raw file to the model. The reduced data may be processed much more quickly than the raw data in commonly applied proteomics tools (prior art methods).

The plurality of characteristics may comprise features extracted from the scan header directly (e.g. Total Ion Current), features extracted from the MS or MS/MS scan (e.g., number of peaks in a scan), features grouped (e.g., if an MS/MS scan is below or equal to the maximal allowed injection time) or calculated values (e.g., Number of MS1 to Number of MS2 scans). The information that is classically applied in prior art methods (i.e., m/z and intensity pairs from MS or MS/MS spectra) may not be required in the proposed methods and it is therefore not essential for these characteristics to be extracted and used as an input for the machine learning model.

The plurality of characteristics may comprise one or more of:
a number of unfragmented scans;
a number of fragmented scans;
a ratio of unfragmented scans to fragmented scans;
a number of peaks in fragmented and/or unfragmented scans;
a median total ion current for fragmented and/or unfragmented scans;
a median ion injection time for fragmented and/or unfragmented scans;
a median charge state value for fragmented and/or unfragmented scans;
a median intensity state value for fragmented and/or unfragmented scans;
a median resolution value for fragmented and/or unfragmented scans;
a median noise value for fragmented and/or unfragmented scans;
a median current, voltage and/or pressure value;
a minimum and/or maximum pressure value;
an automatic gain control fill rate and/or median automatic gain control fill value;
a median baseline value;
a precursor mass to charge ratio;
a precursor signal to noise ratio;
a median scan resolution;
a total ion current isolation window; and
a total ion current for fragmented scans.

Preferably, only representative characteristics are extracted from the raw file and used as an input (both for training the machine learning model and for predicting the sample metric value by a pre-trained machine learning model). In other words, the characteristics used are indicative of LC-MS performance. Sources to extract this representation information may include sections within the raw file, such as the scan header, or from the MS and MS/MS scans.

Characteristics from the raw file may further be grouped into subcategories such as the MSn Level (commonly n=1, 2) or the ion injection time (below or at maximal allowed ion injection time).

Where each raw file is considered as a whole, there may be one input value per characteristic per raw file. Where each raw file is divided into a plurality of retention time slices, there may be one input value per characteristic per retention time slice per raw file.

The machine learning model may be trained using training data obtained from a plurality of different LC-MS combinations. Moreover, the training data may be obtained from one or more LC-MS instruments exhibiting a plurality of different LC-MS performance states (training data may be collected from an LC-MS instrument over a long period of time, so that deterioration in performance of the instrument is represented in the data). By obtaining data from a diverse range of LC-MS combinations and performance states, the performance of the model may be improved.

A data size of the plurality of characteristics may be at least 10 times smaller than a data size of the raw data, preferably 100 times smaller, more preferably 1000 times smaller.

The machine learning algorithm may be used to predict one or more sample metrics (e.g., number of protein groups) using only characteristics related to the raw files as inputs. The machine learning algorithm may only process the plurality of characteristics (i.e., after data reduction to a representative value) and may not directly process the raw data. In other words, the raw data may not be provided as an input to the machine learning algorithm.

The machine learning algorithm may predict the sample metric in less time than prior art methods that rely on database search software (such as Proteome Discoverer).

A time taken to determine the plurality of characteristics from the raw data may be less than a time taken to obtain the raw data by analysing the proteomics sample, preferably at least ten times less.

To reduce the time taken to determine the sample metric (which may be the number of protein groups, number of peptide groups or peptide spectrum matches, as described above), the invention predicts the sample metric by inputting (data reduced) features (that can be extracted quickly from the raw files) into a trained machine learning model.

The plurality of characteristics (features extracted from the raw file) may be extracted from the raw data in a short time (e.g., 5 minutes or less, depending on the size of the raw file and the method applied). The time taken is short compared to the overall runtime of the LC-MS run (which may be approximately 80 minutes in some cases). In this way, processing of the raw data from the proteomics run may be finished quickly so that hardware resources can focus on processing high quality raw files only. If the quality of the raw file is low, it may not be beneficial to invest time and resources with further data processing of the raw files. It may be preferable to skip analysis of the data if the raw file is determined to be of low quality. Alternatively, the sample metric value predicted by machine learning can be used in combination to quality control.

The characteristics may be extracted from the raw files without tying up a significant proportion of the available computing resources, as the process is not resource intensive.

One or more of the plurality of characteristics may be determined from the raw data while the LC-MS obtains the raw data by analysing the proteomics sample (on-the-fly processing). More preferably, all of the plurality of characteristics may be determined from the raw data while the LC-MS obtains the raw data by analysing the proteomics sample.

Thus, the invention provides a fast and efficient method for providing a predicted, high-quality sample related metric value (such as the number of protein groups) for LC-MS systems running proteomics experiments in a controlled environment.

The machine learning model may be a supervised machine learning algorithm. In other words, during training of the model, a sample related metric determined via alternate (more accurate and more time-consuming) means may be provided alongside the training data (the characteristics extracted from the raw training data).

To implement the above methods, training data may be provided. The training data may comprise:
a plurality of characteristics extracted from raw training data; and
one or more corresponding sample metrics obtained (e.g., Number of Protein Groups) from database search software (such as Proteome Discoverer).

Validation data may be provided to optimize the trained machine learning model. The validation data may comprise:
a plurality of characteristics extracted from raw validation data; and
one or more corresponding sample metrics obtained from database search software (such as Proteome Discoverer).

Test data may be provided to assess the trained machine learning model. The test data may comprise:
a plurality of characteristics extracted from raw test data; and
one or more corresponding sample metrics obtained from database search software (such as Proteome Discoverer).

Prediction data may be provided to the trained machine learning model. The prediction data may comprise:
a plurality of characteristics extracted from raw data.

The method may further comprise determining one or more features from the raw data using a database search (such as an ultra-fast database search). Predicting one or more sample metric values may comprise providing the plurality of characteristics and the one or more features to the machine learning model.

A method of training a machine learning model for predicting a sample metric value is provided. The method comprises:
determining a plurality of characteristics from raw training data obtained by analysing a training sample in a LC-MS;
processing the raw training data via a database search to determine the sample metric value for the training sample; and
training the machine learning model using the plurality of characteristics and the sample metric value for the training sample determined via the database search, so that the machine learning model is trained to predict the sample metric value for a subsequent proteomics sample, based on a plurality of characteristics determined from raw data obtained by analysing the subsequent proteomics sample in a LC-MS.

The plurality of characteristics determined from the raw data obtained by analysing the subsequent proteomics sample may be the same characteristics as those determined from the raw training data obtained by analysing the proteomics training sample (e.g., number of MS1 scans, number of MS2 scans, ratio of MS1/MS2 scans, and the like). However, the values of the characteristics will be different between samples.

In some examples, the model may be trained with a subset of the characteristics.

In some examples, a model trained using a particular set of characteristics (“training set”) requires an input value for each of the characteristics in the training set, in order to predict a sample metric value. In some other examples, the model may be used to predict a sample metric value based on values for a subset of the particular set of characteristics (“prediction subset”). In this case, values of the one or more characteristics from the training set that are missing in the prediction subset may be replaced by a dummy value. The dummy value may be zero, a median value from all training data, or another placeholder value. By adding these dummy values to the values of the prediction subset, a set of values comprising a value for each characteristic in the training set may be provided to the model, in order to predict the sample metric value. As some features are only ‘placeholders’, the prediction may be less accurate than if values for each of the characteristics in the training set were provided for the prediction.

The method may further comprise training the machine learning model using one or more of:
features from the LC (e.g., pressure values);
features from one or more calibration files; and/or
features from another database search engine.

The method may further comprise validating the machine learning model by:
determining a plurality of characteristics from raw validation data obtained by analysing a validation sample in a LC-MS;
predicting a sample metric value for the validation sample by processing the plurality of characteristics using the machine learning model;
processing the raw validation data via a database search to determine the sample metric value for the validation sample; and
comparing the sample metric value determined by processing the raw validation data via the database search with the sample metric value predicted by processing the plurality of characteristics using the machine learning model.

The method may comprise determining whether the predicted value (which can be obtained relatively quickly) is sufficiently close to the determined value (which can be obtained accurately but slowly). In other words, the machine learning model may be validated by supplying the trained machine learning model with characteristics obtained from raw validation data to provide a predicted sample metric value and also determining the sample metric value accurately by supplying the raw validation data to a database search engine. The sample metric value may be number of protein groups, Peptide Groups, PSMs, Median of Success Rate, and the like.

The LC-MS method used to collect the training data may be equivalent to the LC-MS method used to collect the validation, test and prediction data.

The LC-MS may be the same LC-MS as used for collecting the training dataset or a different LC-MS. It is not essential that the validation, testing or prediction dataset is collected using the same LC-MS.

Model prediction accuracy may be improved when the data sets (training data, validation data, test data and/or prediction data) come from the same LC-MS instrument and/or using the same standardized LC-MS methods (this is shown in the detailed description below in relation to Applications B, C and D in FIGS. 12, 13 and 14, where different LC-MS system were used but with the same standardized LC-MS methods).

The method may further comprise determining one or more features from the raw training data using a database search (such as an ultra-fast database search). The machine learning model may be trained using the plurality of characteristics, the sample metric value for the training sample determined via the database search and the one or more features determined from the database search. The machine learning model may therefore be configured to predict the sample metric value for a subsequent proteomics sample based on the plurality of characteristics determined from the raw data and one or more features determined from the raw data using a database search.

The step of predicting one or more sample metric values by machine learning can be done using one or more different machine learning models. Examples machine learning models that may be used include Linear Regression, XGBoost, Random Forst, Deep Learning and/or a combination of these (this is shown in the detailed description below in relation to FIG. 4, FIG. 5 and FIG. 7.)

Predicting one or more sample metric values may include predicting a single sample metric (e.g., Number of Protein Groups) or predicting multiple sample metric values simultaneously (e.g., Number of Protein Groups, Number of Proteins, Number of Peptide Groups, Number of Peptide Spectrum Matches, and/or Success Rate) by providing the same sample features/characteristics extracted from the raw file as input data for the one or more trained machine learning models. This is shown in the detailed description below in relation to FIG. 4, FIG. 5 and FIG. 7.

Analysing the proteomics sample in the LC-MS may comprise performing an LC-MS run. The raw data may comprise data relating to a retention time during the LC-MS run. The method may further comprise dividing the retention time into a plurality of retention time slices. The method may further comprise manipulating the raw data to provide data relating to each of the plurality of slices. The plurality of characteristics may comprise a plurality of characteristics for each retention time slice.

As described above, the method may further comprise slicing the raw file into retention time related sections (e.g., 1 minute bins). This approach helps to (i) increase the volume of training data and to (ii) provide more insights into the LC-MS run by providing a predicted value for the Number of Peptide Spectrum Matches (PSMs) at discrete and specific time intervals. This approach includes an additional processing step to obtain the plurality of characteristics. Here, one characteristic is calculated on the specific slice of the raw file (e.g., between retention time 2 and 3 minutes; referred to as ‘local’) but also on the retention time range from the start to the specific time interval (e.g., from retention time 0 to 3 minutes; referred to as ‘global’). This process may be done for the sample metric values and for the plurality of characteristics.

The sample metric value may be used to determine a data quality for raw data obtained by an LC-MS. In other words, the sample metric value may be used to determine an outcome of a data quality test for the LC-MS run, which may be either a “pass” or a “fail”. A data quality outcome should result in a “fail”, when the data quality is low so that further analysis of the raw data is not worthwhile.

A method of determining a data quality for raw data obtained by analysing a proteomics sample in a liquid chromatography mass spectrometer, LC-MS, is also provided. The method comprises predicting one or more sample metric values using the methods described above. The method further comprises, based on the one or more sample metric values, determining whether a data quality for the raw data is above a threshold data quality. If the data quality for the raw data is above a threshold data quality, the method further comprises performing further data analysis on the raw data. Otherwise, if the data quality for the raw data is not above the threshold data quality, the method further comprises skipping further data analysis for the raw data.

The sample metric value may be used to validate a QC run. In other words, the sample metric value may be used to determine an outcome for the QC run, which may be either a “pass” or a “fail”. A QC run should result in a “fail”, when maintenance (or other actions) are required.

A method of determining a quality control, QC, outcome for a liquid chromatography mass spectrometry, LC-MS process is provided. The method comprises, in a QC run, predicting one or more sample metric values by analysing a proteomics sample in a liquid chromatography mass spectrometer, LC-MS, using a method as described above. The proteomics sample is a quality control, QC, sample. The one or more sample metric values comprises a QC sample metric value. The method further comprises, based on the QC sample metric value, determining whether the QC outcome is a pass or a fail.

There are several approaches available to determine when maintenance is required (and therefore whether the outcome should be “pass” or “fail”), based on the QC sample metric value. In one approach, it may be defined that cleaning/maintenance is required when the performance drops by a threshold percentage (e.g., 10%) of the initial performance. In one example, the number of Protein Groups may be used as a QC sample metric value to provide a performance marker/indicator. When the QC sample metric value falls below a threshold (e.g., 90%) of the original value, it may be considered that cleaning/maintenance of the MS is required. The outcome of a QC run may therefore be a “fail” if the QC sample metric value falls below the threshold percentage of the original value (which may be determined in a first QC run following cleaning/maintenance or may be an average of several QC runs, e.g., the first ten QC runs).

There might also be situations in which QC sample metric value suddenly drops by significantly more than the threshold amount. This may be due to an issue or failure in the LC, the sample, or any other part of the MS. In this case, the QC outcome should be a “fail” so that the sequence may be halted, the issue may be fixed and then measurements may be continued in a new sequence. Alternatively, a sudden drop in the QC metric may be an anomaly.

In another approach, the LC-MS measurements may therefore be continued after the QC sample metric value has dropped, in order to determine:
(i) whether the drop in performance is temporary or permanent (e.g., following a gradual decline in performance), and
(ii) whether an unexpected recovery is observed (e.g., following a sudden drop in performance).

As described above, when a QC run result falls below a pre-defined threshold, the QC sample metric value prediction may be repeated in further cycles to check if the measurement of “lower performance” is correct (or whether it was anomalous or recoverable). Further cycles may also be performed after the threshold has been passed for other reasons, as explained below.

If the performance is below the threshold, whether to continue the sequence (issue a “fail” result) or to continue (by issuing a “pass” result) may depend on the level of the deviation of the QC sample metric value below the threshold and the situation. For example when a long-term measurement of biological samples is ongoing and a QC run at the very end of the long term measurement is gradually falling below the threshold, but the deviation below the threshold is nevertheless small, it may be determined that measurements of the “biological samples” should be continued and the maintenance of the LC-MS system is performed after all biological samples have been measured by LC-MS in the same sequence.

However, if the deviation of the QC sample metric value between the result of the QC run and the original level (or threshold level of acceptance) is too high, it may be determined that the sequence of long-term measurements should be interrupted to perform maintenance. Afterwards, the QC run is repeated and evaluated. If the result from the QC sample is passed, the measurement of the biological samples may be continued.

A method of performing a liquid chromatography mass spectrometry sequence is provided. The sequence comprises one or more cycles, each cycle comprising the following steps:
performing a quality control, QC, run and determining a QC outcome via the method described above,
if the QC outcome is a fail, ending the sequence without further cycles; and
if the QC outcome is a pass, performing one or more sample analysis runs (during which biological samples are analysed) and proceeding to the next cycle.

In this method, a cycle is defined as a QC run and one or more sample analysis runs. The sequence continues until a QC run results in a fail outcome. Then, the sequence is terminated, so that maintenance may be performed.

A liquid chromatography mass spectrometer, LC-MS, configured to perform the methods described above is also provided.

Computer software comprising instructions that, when executed by the processor of a computer, cause the computer to perform the methods described above is also provided.

### CLAUSES

The following numbered clauses set out further illustrative examples:
Clause 1. A method of estimating a quality control, QC, metric, the method comprising:
   determining a plurality of characteristics from raw data obtained by analysing a QC sample in a liquid chromatography mass spectrometer, LC-MS; and
   estimating the QC metric by processing the plurality of characteristics using a machine learning algorithm.
Clause 2. The method of clause 1, wherein the QC metric comprises one of:
   a number of protein groups in the QC sample identifiable by the LC-MS;
   a number of peptide groups in the QC sample identifiable by the LC-MS; and
   a number of peptide spectrum matches in the QC sample identifiable by the LC-MS.
Clause 3. The method of clause 1 or clause 2, wherein the plurality of characteristics comprise one or more of:
   a number of unfragmented, MS1, scans;
   a number of fragmented, MS2, scans;
   a ratio of unfragmented scans to fragmented scans, MS1/MS2;
   a number of peaks in unfragmented scans;
   a number of peaks fragmented scans;
   a median total ion current for unfragmented scans;
   a median total ion current for fragmented scans;
   a median ion injection time for unfragmented scans;
   a median ion injection time for fragmented scans;
   a median charge state value for unfragmented scans;
   a median charge state value for fragmented, MSn, scans;
   a median intensity state value for unfragmented scans;
   a median intensity value for fragmented, MSn, scans;
   a median resolution value for unfragmented scans;
   a median resolution value for fragmented, MSn, scans;
   a median noise value for unfragmented scans;
   a median noise value for fragmented, MSn, scans;
   a median current value;
   a median voltage value;
   a median pressure value;
   a minimum pressure value;
   a maximum pressure value;
   an automatic gain control fill rate;
   a median automatic gain control fill value;
   a median baseline value;
   a precursor mass to charge, m/z, ratio;
   a precursor signal to noise, S/N, ratio;
   a median scan resolution;
   a total ion current, TIC, isolation window; and
   a total ion current, TIC, for fragmented scans.
Clause 4. The method of any preceding clause, wherein a data size of the plurality of characteristics is at least 10 times smaller than a data size of the raw data, preferably 100 times smaller, more preferably 1000 times smaller.
Clause 5. The method of any preceding clause, wherein the machine learning algorithm only processes the plurality of characteristics and does not directly process the raw data.
Clause 6. The method of any preceding clause, wherein a time taken for the machine learning algorithm to estimate the QC metric is less than a time taken for a Proteome Discoverer algorithm to determine the QC metric based on the raw data.
Clause 7. The method of any preceding clause, wherein a time taken to determine the plurality of characteristics from the raw data is less than a time taken to obtain the raw data by analysing the QC sample, preferably at least ten times less.
Clause 8. The method of any preceding clause, wherein one or more of the plurality of characteristics are determined from the raw data while the LC-MS obtains the raw data by analysing the QC sample.
Clause 9. The method of any preceding clause, further comprising:
   determining one or more parameters from an ultra-fast database search, based on the raw data,
   wherein estimating the QC metric using a machine learning algorithm comprises providing the plurality of characteristics and the one or more parameters to the machine learning algorithm.
Clause 10. A method of training a machine learning algorithm for estimating a quality control, QC, metric, the method comprising:
   determining a plurality of characteristics from raw training data obtained by analysing a QC training sample in a LC-MS;
   processing the raw training data via a database search to determine the QC metric for the QC training sample; and
   training the machine learning algorithm using the plurality of characteristics and the QC metric for the QC training sample determined via the database search, so that the machine learning algorithm is configured to estimate the QC metric for a subsequent QC sample, based on a plurality of characteristics determined from raw data obtained by analysing the subsequent QC sample in a LC-MS.
Clause 11. The method of clause 10, further comprising validating the machine learning algorithm by:
   determining a plurality of characteristics from raw validation data obtained by analysing a QC validation sample in a LC-MS; and
   estimating a QC metric for the QC validation sample by processing the plurality of characteristics using the machine learning algorithm;
   processing the raw validation data via a database search to determine the QC metric for the QC validation sample; and
   comparing the QC metric determined by processing the raw validation data via the database search with the QC metric estimated by processing the plurality of characteristics using the machine learning algorithm.
Clause 12. A method of determining a quality control, QC, outcome for a liquid chromatography mass spectrometry, LC-MS process, the method comprising:
   in a QC run, estimating a QC metric by the method of any of clauses 1 to 8; and
   based on the QC metric, determining whether the QC outcome is a pass or a fail.
Clause 13. A method of performing a liquid chromatography mass spectrometry sequence, the sequence comprising one or more cycles, each cycle comprising the following steps:
   performing a quality control, QC, run and determining a QC outcome via the method of clause 12,
   if the QC outcome is a fail, ending the sequence without further cycles; and
   if the QC outcome is a pass, performing one or more sample analysis runs and proceeding to the next cycle.
Clause 14. A liquid chromatography mass spectrometer, LC-MS, configured to perform the method of any preceding clause.
Clause 15. Computer software comprising instructions that, when executed by the processor of a computer, cause the computer to perform the method of any of clauses 1 to 13.

### BRIEF DESCRIPTION OF THE DRAWINGS

FIG. 1 illustrates a schematic diagram of a mass spectrometer.

The invention will be described with reference to the non-limiting examples illustrated in the following Figures.

FIG. 2 illustrates for each QC run in a test dataset, the number of normalized protein groups predicted via the proposed methods, as well as the number of protein groups determined by processing the raw data via a database search. The same LC-MS instrument was used for collecting the training and test dataset.

FIG. 3 illustrates for each QC run in a test dataset, the number of normalized protein groups predicted via the proposed methods algorithm, as well as the number of protein groups determined by processing the raw data via a database search. Two different LC-MS instruments were used for collecting the training and test dataset.

FIG. 4 illustrates for each raw file in a test dataset, the number of normalized protein groups predicted via three proposed machine learning models, as well as the number of normalized protein groups determined by processing the raw data via a database search. Various different LC-MS instrument combinations were used for collecting the training and test dataset.

FIG. 5 illustrates for each raw file in a test dataset, the number of normalized protein groups predicted via an average of three machine learning models, as well as the number of normalized protein groups determined by processing the raw data via a database search. Various different LC-MS instrument combinations were used for collecting the training and test dataset.

FIG. 6 illustrates the distribution of the relative error between the predicted number of protein groups and the number of protein groups determined by processing the raw data via a database search. The error was rounded to one digit.

FIG. 7 illustrates, for each raw file in a test dataset, the number of normalized peptide spectrum matches (PSMs) predicted via an average of three machine learning models, as well as the number of normalized peptide spectrum matches (PSMs) determined by processing the raw data via a database search. Various different LC-MS instrument combinations were used for collecting the training and test dataset.

FIG. 8 illustrates the distribution of the relative error between the predicted number of peptide spectrum matches and the number of peptide spectrum matches determined by processing the raw data via a database search. The error was rounded to one digit.

FIG. 9 illustrates, for a single and high-quality raw file in a test dataset, the number of normalized peptide spectrum matches predicted via a machine learning model (multiple linear regression), the number of normalized peptide spectrum matches predicted via an average of three machine learning models, and the number of normalized peptide spectrum matched determined by processing the raw data via a database search (Proteome Discoverer) for specific time intervals (1 minute). Various different LC-MS instrument combinations were used for collecting the training and test datasets.

FIG. 10 illustrates, for a single and very low quality raw file in a test dataset, the number of normalized peptide spectrum matches predicted via a machine learning model (multiple linear regression), the number of normalized peptide spectrum matches predicted via an average of three machine learning models, and the number of normalized peptide spectrum matched determined by processing the raw data via a database search (Proteome Discoverer) for specific time intervals (1 minute). Various different LC-MS instrument combinations were used for collecting the training and test dataset.

FIGS. 11A to 11D illustrate data obtained during four different sequences run on the same LC-MS setup. These Figures illustrate, for each QC run in the sequence, the (normalized) number of protein groups obtained by processing the raw data with a database search.

FIG. 12 illustrates, for each QC run in a validation dataset, the (normalized) number of protein groups predicted via the proposed methods, as well as the (normalized) number of protein groups determined by processing the raw data via a database search.

FIG. 13 illustrates, for each day during which data were collected in the validation dataset, the (normalized) average number of protein groups predicted by the machine learning model, compared to the (normalized) average number of protein groups determined by processing the raw data via a database search.

FIG. 14 illustrates how the proposed methods perform on a validation dataset obtained during a sequence run on a different LC-MS setup. FIG. 14 illustrates the predicted (normalized) number of protein groups by machine learning and the (normalized) number of protein groups determined by processing the raw data via a database search. Here, as with FIG. 13, multiple results from a single day are averaged and shown as a single data point for a single day.

FIG. 15 illustrates how the proposed methods perform on a validation dataset obtained during a sequence run on a third LC-MS setup. FIG. 14 illustrates the predicted (normalized) number of protein groups by machine learning and the (normalized) number of protein groups determined by processing the raw data via a database search. Here, as with FIGS. 13 and 14, multiple results from a single day are averaged and shown as a single data point for a single day.

FIG. 16 illustrates a graph of the (normalized) number of protein groups predicted/determined from validation data. The predicted (normalized) number of protein groups from two machine learning algorithms is compared with the (normalized) number of protein groups determined by processing the raw data via a database search (a thorough database search). In the first machine learning model, the inputs to the ML model are features from a fast database search. In the second machine learning model, the inputs to the ML model are features from a fast database search and characteristics extracted from the raw data.

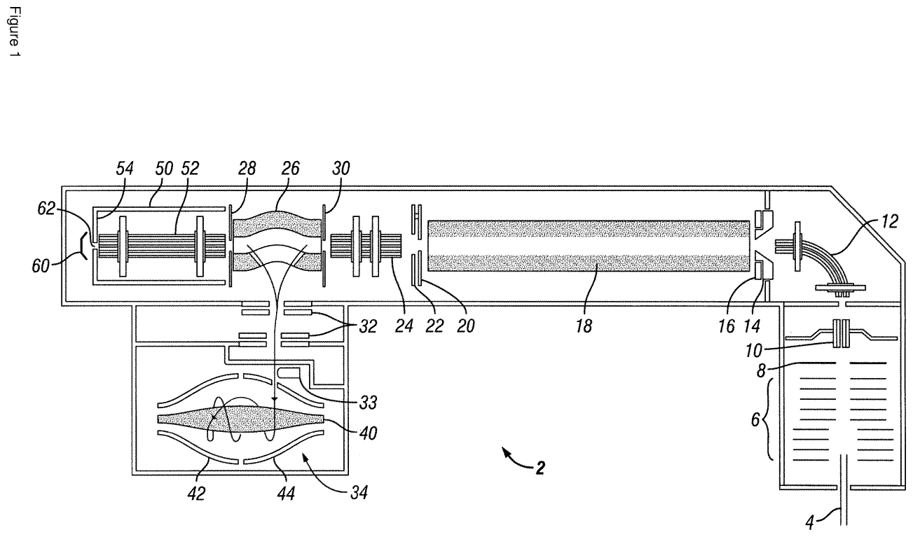

### DETAILED DESCRIPTION

The most applied sample metric to evaluate sample runs (including QC runs) in proteomics labs is the sample metric “Total Number of Protein Groups (identified)”. The value of this sample metric is a combination of various other sample metrics (Peptide Groups, Peptide Spectrum Matches (PSMs)) and filter options, which makes the value of this sample metric relatively robust (and therefore makes this a suitable sample metric for application in QC). Although this sample metric is a combination of several features, a decrease in the value of the number of protein groups sample metric is an indicator that the LC-MS system (including samples and solvents) does not perform as expected when analysing a known sample with a standardized LC-MS method.

A value for the number of protein groups sample metric can be obtained by running a database search using software such as Proteome Discoverer (“PD”). As running Proteome Discoverer is a time and computing resource intensive process, an alternative solution is proposed, which utilizes Supervised machine learning to predict one or more sample metric values (such as the Number of Protein Groups) by using only characteristics (features) extracted from the raw file. These raw file related features can be extracted from the raw file in a relatively short time (preferably less than 5 minutes) compared to the overall runtime of the LC-MS run (approximately 80 minutes in some cases). The process of extracting the characteristics from the raw file is not hardware-resource intensive and can be run while the LC-MS system continues to acquire raw files.

In an ideal situation, the plurality of characteristics are extracted while the proteomics sample (for example a QC sample) is still being analysed (on the fly processing). The final calculation of the median, relative or aggregated values used as input values for the machine learning model is typically a fast process. Also, the prediction of the protein groups (or any other commonly applied sample metric) by machine learning is very fast.

In an ideal situation (with on the fly processing of the QC run), the software controlling when a new sample is injected for analysis by LC-MS would (i) only need to wait a very short time (e.g., <1 minute) to get a predicted sample metric value (e.g., Number of Protein Groups) reported by a trained machine learning model. Alternatively (or additionally), (ii) the Number of Peptide Spectrum Matches (PSMs) for one or more specific retention time range is predicted by machine learning.

Depending on the value of the sample metric predicted by machine learning, this can be rated as a positive or negative event. This event is provided to the instrument control software. Before this response is provided, no further sample would be injected (where the sample injection is controlled by the software, which is not essential).

Alternatively, in other proposed examples, the control software may either:
(i) inject a new biological sample before the response is provided. This sample may be wasted if the run is aborted in the event of a negative response) or
(ii) wait for a user input (the user may manually check the result of the QC run and enter a result) indicating whether to proceed with the analysis of “biological” samples or to interrupt the sequence.

To train the machine learning model and validate its performance to predict a sample metric value (e.g., the number of protein groups identified), at least two different data sets are required in these examples:

### 1. Training Data Set

This dataset contains the extracted information from raw files plus the identification results from a database search software (such as Proteome Discoverer). It is used to train the machine learning model.

### 2. Test Data Set

This dataset contains the extracted information from raw files which are used to predict the number of protein groups (or any other sample metric) by the previously trained machine learning models. The test data set also contains information from the database search, to evaluate the results obtained by the machine learning model. This is a useful step during the development to prevent misinterpretation of the sample metric values predicted by machine learning.

### 3. (Optional) Validation Data Set

This dataset contains the extracted information from raw files plus the identification results from a database search software (such as Proteome Discoverer). It is used to optimize the machine learning model. Often, this dataset is a subset of the training dataset (and eliminated from the training dataset).

Once the machine learning algorithm has been trained and evaluated, it may be used to process “live” data (for example, data from a QC run where an estimate of the QC metric is required) and predict the number of protein groups (or any other sample metric). In this case, identification results from a database search software are not available at this stage.

### 4. Prediction Data Set

In its final application, information obtained from the database search is not required. Solely, the raw file related information from an LC-MS run (using the same sample and LC-MS method) is used to predict one or multiple sample metric values (e.g., the Number of protein groups) by machine learning.

Proteomics raw files (e.g., obtained from QC runs) can contain MS1 and MS2 spectra, with hundreds of MS peaks per scan. The proposed methods reduce the complexity of the data prior to processing to a least necessary minimum. This data reduction may be done by reporting median values, absolute values and/or relative values, as described above.

Reducing the complexity of the raw data facilitates creation of the plurality of characteristic values for the test and training data. The volume of data requiring processing to predicted the sample metric value is reduced by eliminating the retention time domain in some examples. In other examples, the raw file may be sliced into specific retention time windows and characteristics may be extracted from each of the retention time windows. The retention time domain information is also removed from this reduced data set.

In some examples, there are hundreds of features in the test and training datasets, which can be extracted from raw file data (depending on the LC and MS utilized). The sample metrics to be predicted can be delivered by a database search engine. All features used to train and process the ‘unseen’/new sample can be grouped into subcategories:

a. Identification Results from a Database Search Engine

Examples: Protein Groups, Peptide Groups, Number of Peptide Spectrum Matches (PSMs), Success Rate (=Number of Peptide Spectrum Matches/Number of MS2 scans)

b. Absolute and Relative Values from the QC Run

Examples: Number of MS1 Scans, Number of MS2 Scans, Ratio of Scans (MS1/MS2)

c. Median Values from Ion Transmission

Examples: TIC MS1, Ion Injection Time MS1, TIC MS2, Ion Injection Time MS2

d. Median Values from MSn Scans

Examples: Charge States, Intensity, Resolution, Noise

e. Median Values from Status Log

Examples: Current and Voltages from Boards installed in the MS

f. Median, Minimal and Maximal Values for the LC

Examples: Pressure values

Values from sub-category a) are provided in the training, validation and test data sets and used to train, optimize, and test the predictions made by the machine learning model. For predicting sample metrics (such as the Number of Protein Groups), these values are usually not available as it can be time intensive to obtain these from the database search engines (as described above).

Many prior art search engines rely only on the MS2 peaks and intensities. Therefore, the proposed approach is superior in contrast to prior art methods at least because more information extracted from the raw data is used.

In some prior art methods, QC runs are evaluated using database search engines and the LC-MS setup is validated based on the total number of protein groups. The database search is a very computer resource and time intensive approach. To reduce the time and hardware resources required, the proposed methods utilise machine learning to predict a sample metric value (such as the number of protein groups) using only characteristics extracted from the raw data. These characteristics can be extracted from the raw file within a relatively short time and with less hardware resources than a full database search. This enables determination of the state of the LC-MS system by predicting a discrete value (e.g., Number of Protein Groups) shortly after the QC run has completed. Furthermore, the characteristics extracted from the raw file are orthogonal to the search engine results, since the characteristics extracted from the raw file relate to the entire LC-MS system, whereas the search engine results relate (mainly) to the MS2 data quality.

In prior art methods, a relatively small number of QC runs may be performed during the peak performance period of the instrument, in order to determine a lower limit of the number of protein groups before a QC run is marked as “failed” (a threshold). The deviation between the QC runs may be calculated as a Median Absolute Deviation (MAD). This MAD value may be multiplied by a factor (e.g., a factor of 2 or 3) to set the lower warning level. However, this approach only focuses on a short time frame.

In contrast to the prior art approach, examples of the proposed approach provide a training dataset that covers various stages (such as time course experiments and/or different instruments) of the LC-MS setup, in order to train the ML algorithm. In other words, data is obtained while the setup is at its top performance but also when the LC-MS setup (1) differs from the expectation or (2) differ in its nature (i.e., different MS from the same model series), in order to make a good prediction about the quality of the raw files. Nevertheless, once trained, the ML model can provide the results of a QC run more quickly than prior art approaches (in real-time/on-the-fly in some examples).

In some examples, two data reduction steps are performed to reduce the data complexity:

i. Reducing a Parameter to a Single Value

Raw LC-MS data contains a m/z domain and a (retention) time domain, as the samples are separated by LC and the peptides elute based on their physico-chemical properties. When all parts of the LC-MS setup perform as expected, representing a feature by its average or median value is valid approach, as this value should not change when the LC-MS setup is unchanged. Any change represents a deviation which can negatively affect an LC-MS setup.

Alternatively, the raw file may be sliced into smaller pieces based on the retention time and representative values for these slices might be extracted.

ii. Reducing the Number of Features

The overall number of features is reduced from up to several hundred (which are available via the raw file) to only a subset of relevant characteristics (around 30 characteristics in the specific examples described below), which can effectively represent the performance of an LC-MS setup. During the training of the machine learning model, it turns out that only this subset of characteristics is needed to predict the sample or QC metric accurately. Indeed, using this subset of characteristics as machine learning inputs was found to result in the machine learning model performing particularly well (both in terms of accurate prediction and also fast processing time). Adding more features may or may not improve the prediction results. In some cases, the predicted results may be negatively affected by adding more features (for example, when readback or calibration values do not correlate with the number of protein groups).

As discussed above, the number of protein groups is a useful parameter to evaluate QC runs. In prior art database methods, the number of protein groups is based (including some downstream processing to build protein groups starting from PSMs via Peptide Groups via Proteins) on the MS2 data quality. The process to match experimental and theoretical peptide spectra to obtain Peptide Spectrum Matches is a hardware and time intensive process.

In contrast, predicting the protein groups (or any other sample related metric) for proteomics LC-MS data in a controlled environment (i.e., having a standardized LC-MS method) by machine learning is only based on some key characteristics extracted from a raw file. Mostly, between 30 and 40 characteristics are used (depending on the Mass Spectrometer used), instead of hundreds of features that are available in the raw files that can be considered as key features. This reduction in data complexity improves the processing of the data. In terms of quality control, this allows the instrument control software to decide quickly if the sequence needs to be interrupted (or not), based on the sample metric value predicted by Machine Learning. Since the next run may not commence until the sample metric value has been determined (sample could be wasted if it is injected prior to determining a “fail” outcome from the immediately previous QC run). Time spent processing the data from the QC run may delay the workflow for the remainder of the sequence (in other words, processing the data from the QC run can create a bottleneck). Preferably, the QC outcome may be determined before the system is ready to start the next run (which may be a production run). In some examples, the data from the QC run is analysed while further data from the same run is still being collected (on-the-fly processing). The proposed methods provide clear advantages over classical methods (e.g., database search engines), as the sample metric value(s) can be predicted very fast.

### Experimental Data

In this section, two different datasets are used to demonstrate the three example applications of using machine learning to predict sample metric values (such as the number of protein groups). The input into the machine learning models comprises extracted characteristics and representative features from raw files. These features are highly reduced from the raw data to keep the approach “lightweight” and allow the sample metric values to be predicted quickly.

I. QExactive HF-X Data from Long-Term Measurements

The first dataset consists of a long-term measurement performed on a QExactive HF-X coupled to an Ultimate 3000. Here, standardized measurements (i.e., using the same sample and LC-MS method) were injected over a long period of time as a quality control sample among real biological samples.

The training dataset (referred to as ‘Instrument 06’) consisted of 400 raw files. Each single raw file delivered a single sample metric to predict, which is the number of Protein Groups in this example. Additionally, 77 features (i.e., 77 representative values for a single raw file) were extracted from the raw file which were related (and variations to): number of spectra, number of peaks per spectra, ion injection times, ion fluxes and intensities, charge states, signal to noise, information about the precursor and information if enough ions were present for collecting a scan. Variations of these features can relate to MS1 or MS2 but also to relative values by setting one feature into relation to another. Approximately half of these features were obtained by grouping the features further into a subcategory. In this example, it was the ion injection time (below or at maximal ion injection time) which was used for grouping.

The first test dataset was acquired on the same LC-MS setup (Instrument 06) using the same environment (i.e., the LC-MS method was kept constant). Consumable, such as the sample and the LC-column were kept as constant as possible. This dataset is referred to as ‘Instrument 06b’ in these examples and consists of 404 raw files. The features were extracted in a similar manner as for the training dataset, as described above.

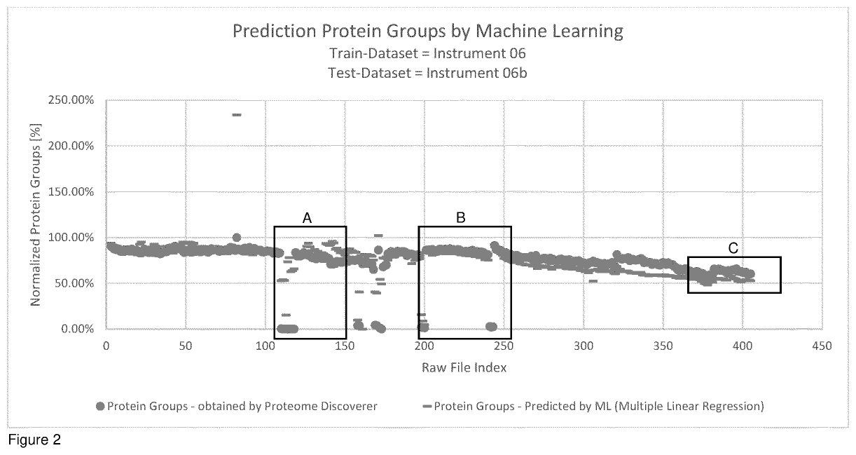

For visualization, the protein groups were normalized to the highest value obtained by Proteome Discoverer. The predictions made by a trained machine learning model are shown in FIG. 2. For the first 100 raw files (approximately), the prediction was very accurate. Within the range of raw files 100 and 150 (approximately), the prediction was less accurate (labelled with ‘A’).

The prediction was more accurate again within the approximate range of raw files 200 and 250 (labelled with ‘B’). Notably, a decrease in performance to 60% normalized protein groups is observed between raw files 245 and 400 (approximately), as predicted—although the prediction was showing lower values compared to the true values (labelled with ‘C’).

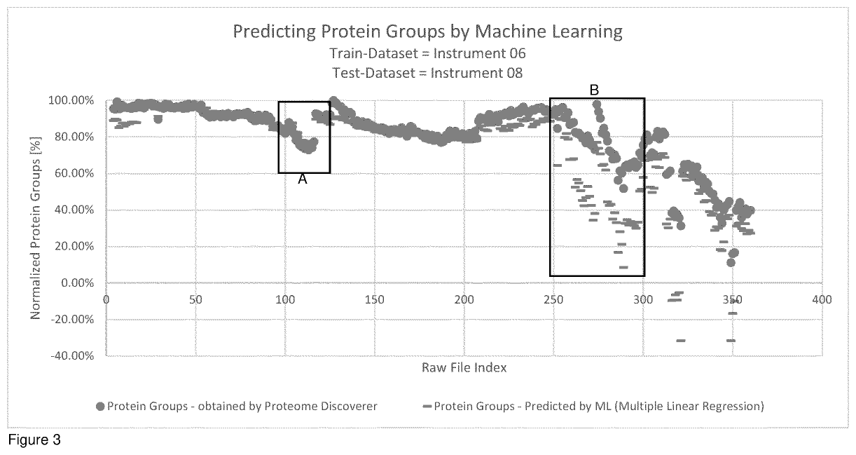

A second test-dataset was collected using the same environment (i.e., same LC-MS method and keeping the consumables as constant as possible), but a different LC-MS (Instrument 08). Using the same trained machine learning model, the predicted data was very precise until raw file index 250 (see FIG. 3). Even smaller drops in performance (e.g., around raw file index 110; see label ‘A’) could be predicted accurately.

The general curve of decreasing performance could also be predicted for the raw files with raw file index 250 to 350. For some specific regions, the prediction was much lower than the true value obtained by Proteome Discoverer (labelled with ‘B’). Towards the end of the dataset, the prediction was again closer to the true value.

II. Orbitrap Exploris 480 Data from Various LC-MS Combinations

The application of machine learning to predict a sample metric (e.g., Number of Protein Groups) shown above was based on training a machine learning algorithm on raw files (the training dataset), which were collected using the same LC-MS setup (Instrument 06) over a long period of time. It was already shown that trained models can also be used to predict sample metrics from different instruments utilizing the same LC-MS method.

In a different example, which is described below, the training data was obtained from various LC-MS combinations. For all data, the LC-MS method used for acquiring the raw files was kept constant.

The entire dataset comprises approximately 2500 raw files. 80% of these raw files were used to train the machine learning algorithms and 20% were used for testing.

The approach differs from the approach described previously in how the input/training data was created (using various different LC-MS setups), and also in performing prediction by machine learning. In this example, the sample metrics ‘Number of Proteins’, ‘Number of Protein Groups’, ‘Number of Peptide Groups’, ‘Number of Peptide Spectrum Matches’, and the ‘Success Rate [Number of Peptide Spectrum Matches/Number of MS/MS scans]’ were predicted simultaneously by three different machine learning algorithms (Multiple Linear Regression, XGBoost and Random Forest). This is different to the previous example (Long-Term Test using two QE HF-X instruments) where only the number of protein groups was predicted by machine learning using multiple linear regression.

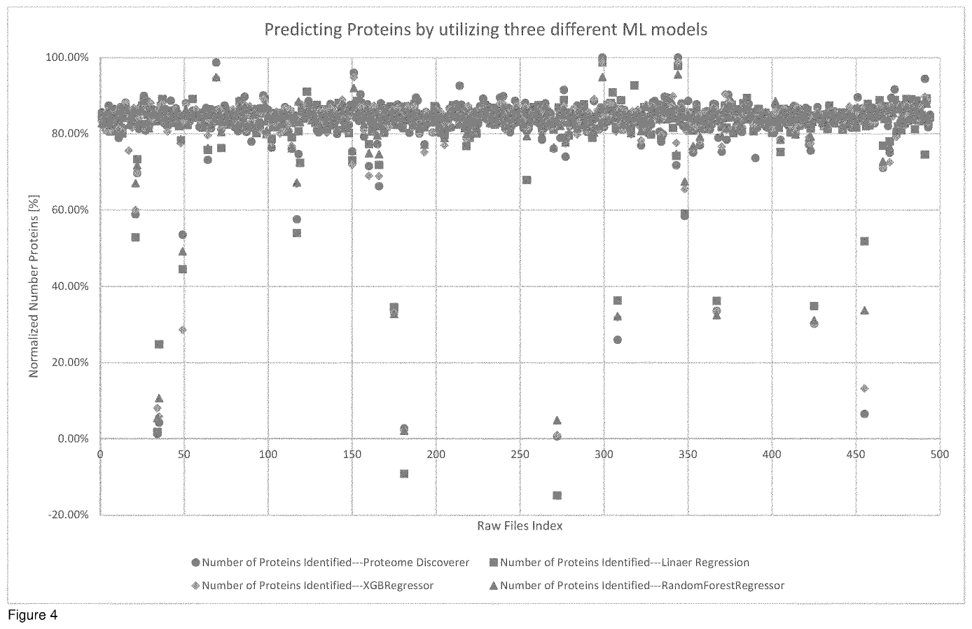

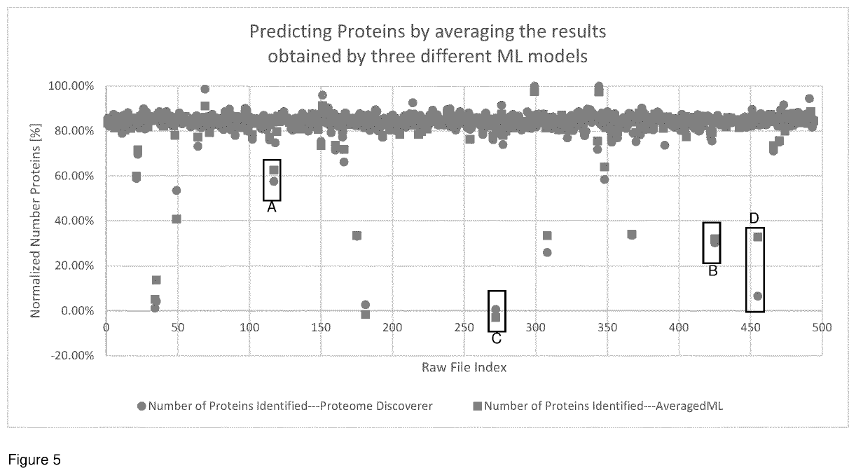

The prediction for three trained machine learning models (Multiple Linear Regression, XGBoost and Random Forest) compared to the results obtained by Proteome Discoverer is shown in FIG. 4. In this graph, the ‘Number of Proteins’ are normalized to 100% using the highest value in the dataset. To reduce variations obtained from individual machine learning predictions, the results obtained were averaged (‘AveragedML’ in FIG. 5). Although most of the data was located at 80-90% (relative scale to highest value), also values located at 60, 30 or close to 0% could be predicted accurately (labelled with A, B and C in FIG. 5.

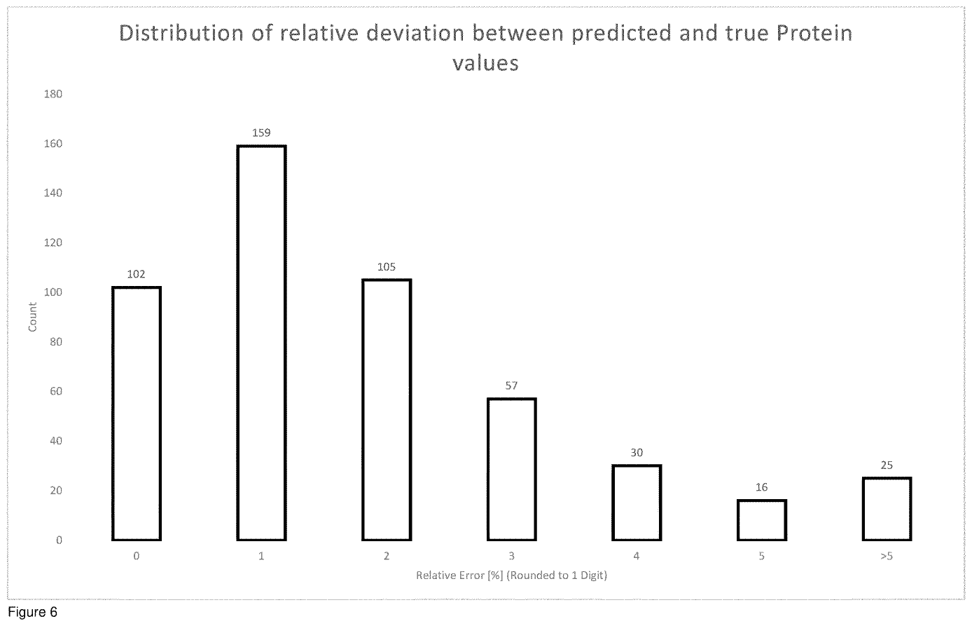

The mean absolute error for the ensemble of predictions, in relation to the true value reported by Proteome Discoverer, was approximately 5%. The median was calculated to be 1.3%. As indicated in FIG. 6, for 95% of the data, the prediction was very accurate (up to and including 5% relative error). Consequently, 5% of the predicted values showed a very high deviation (25 predictions were off by more than 5%). In some rare cases, the relative mean absolute error was much higher (>100%). An example is labelled with ‘D’ in FIG. 5. Here, the relative deviation was approximately 400%.

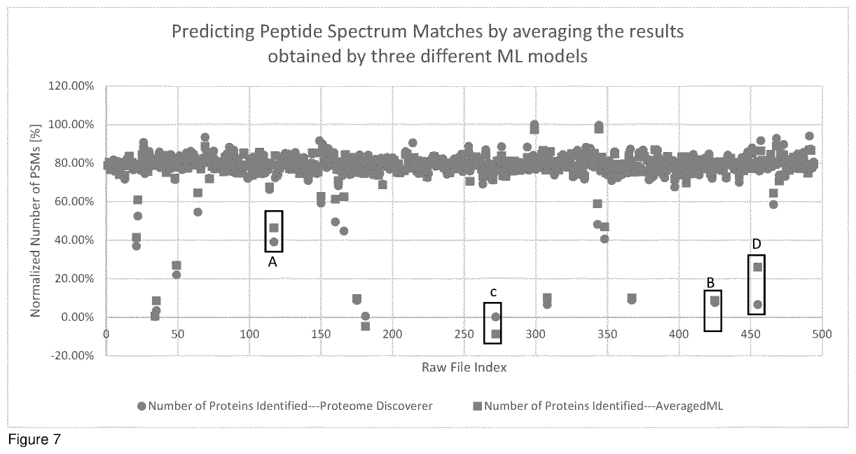

For the ‘Number of Peptide Spectrum Matches’ (PSMs), the average predictions from three machine learning models are shown in FIG. 7. Overall, (as illustrated by the predictions labelled with A, B and C) the sample metric values predicted by the machine learning models are close to the “true” values obtained from Proteome Discoverer. Again, the datapoint labelled with ‘D’ represents an example, where the prediction differs significantly from the “true” value.

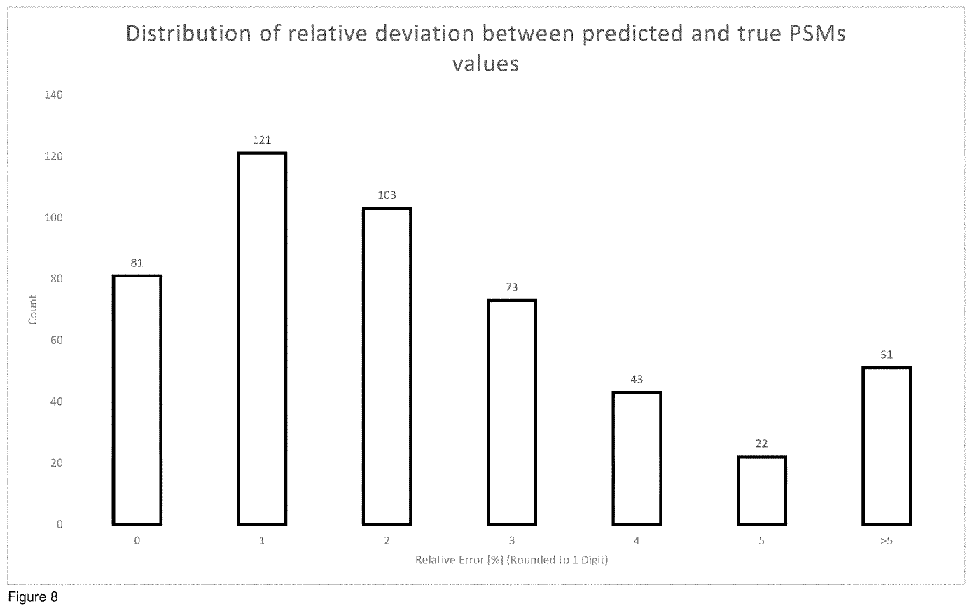

As shown before, the average relative error is skewed by some ‘extreme’ outliers. In this case, the median absolute error was calculated to be 2%. This is slightly higher compared to the number of proteins. As described above for predicting for the Number of Proteins, individual predictions can differ and show a higher deviation (51 predictions were off by more than 5%, as can be seen by comparing FIG. 8 to FIG. 6). Especially for low quality runs (i.e., runs with a relative PSM value of 0.1% or lower), the deviation between true and predicted value was very high.

III. Orbitrap Exploris 480 Data from Various LC-MS Combinations to Predict Peptide Spectrum Matches Per Retention Time Interval

In the previous section, multiple sample metric values (such as the number of protein groups) were predicted by multiple trained machine learning models. Some example methods comprise extracting a representative value for each characteristic or “feature” from a raw file. In other words, if 30 features were extracted from a raw file, the raw file was described by these 30 features.

In order to benefit from other data, which is available in the raw file (but nevertheless reducing the data size and processing time significantly compared to Proteome Discoverer), an alternative approach is presented below.

This approach slices the raw file into specific retention time windows and extracts two representative values for each slice and feature. In the case of 30 features extracted (such as the Number of MS1 scans) for a single raw file, a raw file is described by 60 features per slice. These are comprised of 30 “local” features (relating to a specific retention time slice) and 30 “global” features (relating to the period from the start up to and including this retention time slice). Additionally, the number of input data is increased by a factor calculated as “LC-MS Run Time divided by Retention Time Window Size”. The information about the retention time is removed from the training and test dataset.

The sample metric Peptide Spectrum Matches is well-suited to be used in this retention time slicing approach, as the sample metric PSM is directly linked to a particular MS/MS spectrum.

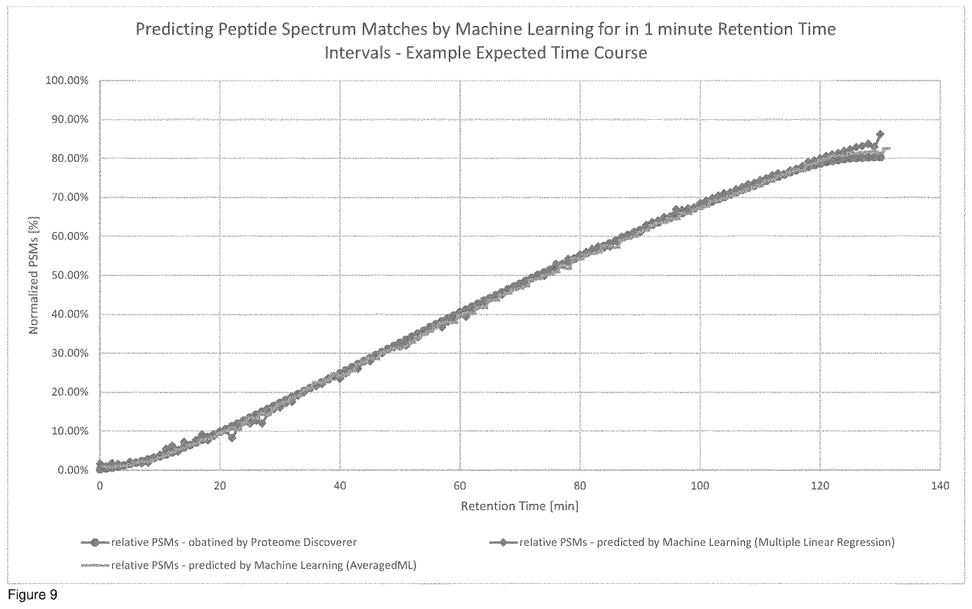

In FIG. 9, the PSMs predicted and obtained by Proteome Discoverer in 1 minute intervals are shown. The PSMs were normalized to the highest value in the training dataset. The PSMs predicted for each time interval match very well with the “true” value obtained by Proteome Discoverer. This is true for a single machine learning model using multiple linear regression and also for the averaged machine learning outcome from three models (referred to as ‘AveragedML’).

This example represents a commonly seen curve shape, as seen by the final relative PSM value of approx. 80%.

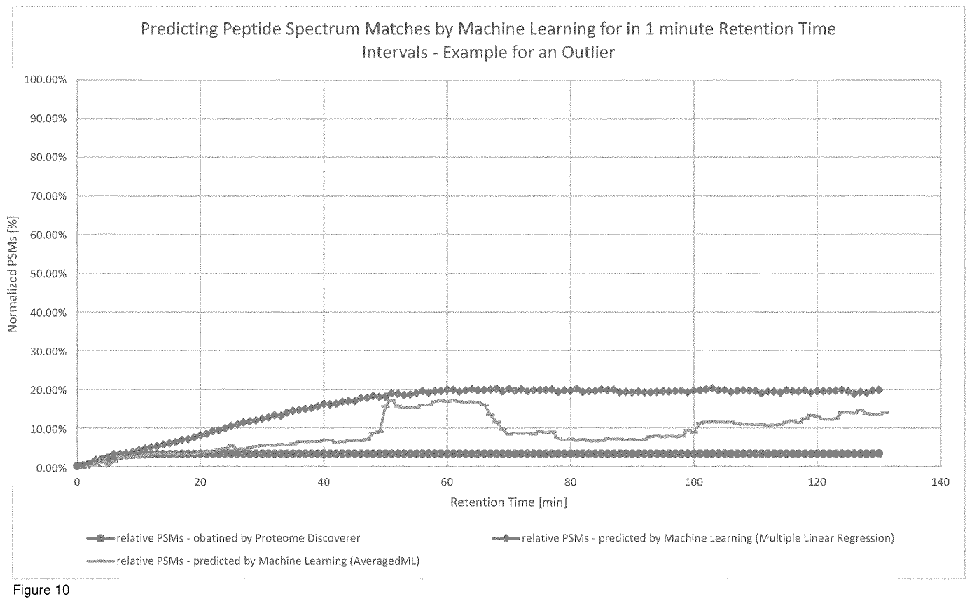

The prediction accuracy is lower when ‘outliers’ in relation to the overall training data are predicted. This is shown in FIG. 10 illustrating normalized PSM against retention time, which forms a curve with a final relative PSM value of 3.5%, compared to the highest PSM contained in the training dataset. The machine learning model ‘multiple linear regression’ predicted a final relative PSM value of 20%, which was much higher than the true value (3.5%).

By averaging the three trained machine learning models (‘AveragedML’), the final prediction was at 13%. Although the difference between the true PSM value obtained by Proteome Discoverer and machine learning is very different, the results still indicates that this raw file is of lower quality. This can be particularly useful when analysing QC samples to get fast feedback on the LC-MS setup performance.

### IV. Variations to the Features Extracted and Used for Machine Learning and the Optimal Training Dataset

Lastly, variations to the training set and the corresponding features extracted from the raw files, respectively, to predict sample specific metrices (e.g., the number of protein groups, or Peptide Spectrum Matches) may also contain features which cannot be read directly from the raw file. This may cover QC data from classical evaluation processes (a priori). Such data may relate to the LC (e.g., as the effective gradient length, FWHM, retention time stability of spiked in peptides, such as Thermo PRTC, or pump pressure profiles) or results from a different (but faster) search algorithm/engine. This makes the overall approach of using machine learning to predict a sample metric in a controlled environment (i.e., by keeping the LC-MS method constant) flexible. As for each machine learning project, the training data is of high importance. Ideally, the dataset is collected using different LC-MS combinations which were run over a long period of time to get training data at its peak performance but also at lower performance.

### Further Experimental Data

For further demonstrating the feasibility of using characteristics extracted from the raw file to predict a sample metric value by machine learning, a selection of data was obtained using three LC-MS systems (OE 480 MS & Ultimate 3000; named M01, M02 and M03). The systems were operated under controlled and defined conditions using the same LC-MS methods.

All LC-MS setups were operated 24 hours a day and seven days a week. The sequences consisted of Plasma samples to contaminate the LC-MS setup, while HeLa samples were measured as QC runs to demonstrate how long such a setup can operate until a cleaning of the LC-MS system is required.

Each LC-MS setup was operated at least once until a cleaning\maintenance of the MS was required. This time range is referred to as a “sequence”.

The first of the three LC-MS instrument setups (M01) was utilised to run four data acquisition sequences (M01a, M01b, M01c and M01d).

In each of the four experiments described below, the machine learning algorithm was trained using data from the first three of the four sequences from M01: M01a, M01b and M01c.

In a first experiment, the data from the first three sequences of the first MS (M01a-M01c) were used to train a machine learning algorithm, which was then validated using the data from the fourth sequence from the first MS (M01d). For each QC run in the fourth dataset, a plurality of characteristics were extracted from the raw data, which were then supplied as inputs to the ML algorithm and used to predict the number of protein groups for each QC run in the fourth dataset. The results were compared against the number of protein groups determined by analysing the raw data using the Proteome Discoverer software.

In other words, for the first experiment, the training dataset consists of three sequences of M01 (namely: M01a, M01b, M01c). Data obtained during the last sequence (M01d) was used as test dataset to evaluate how well the trained machine learning algorithm can predict protein groups from the same LC-MS setup using only characteristics extracted from the raw data (rather than processing the entire raw data).

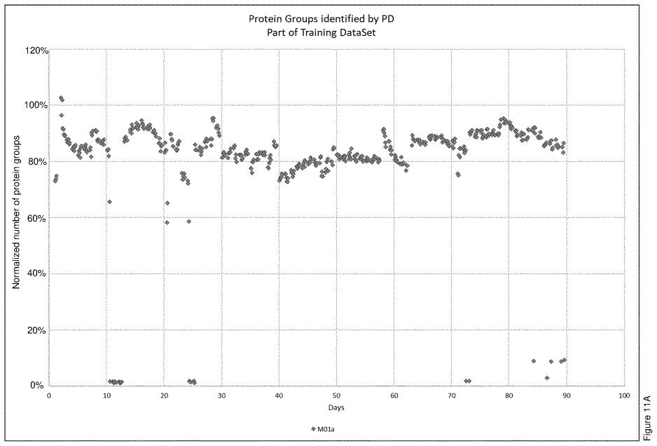

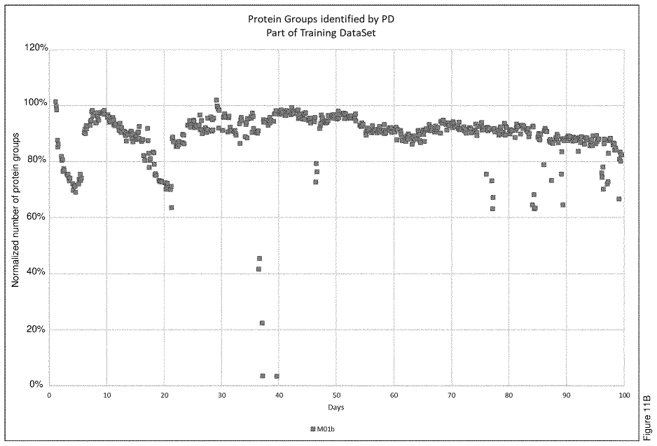

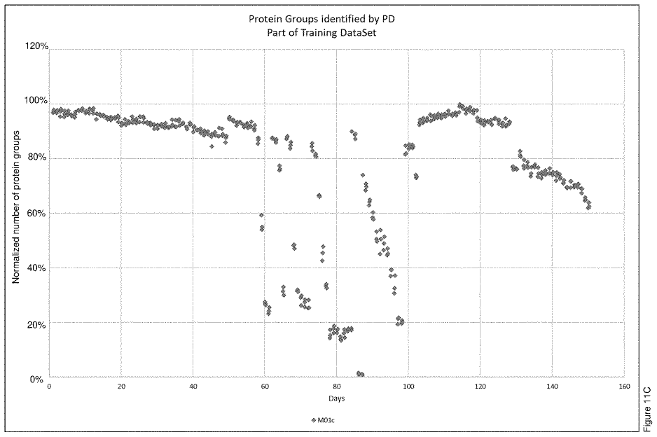

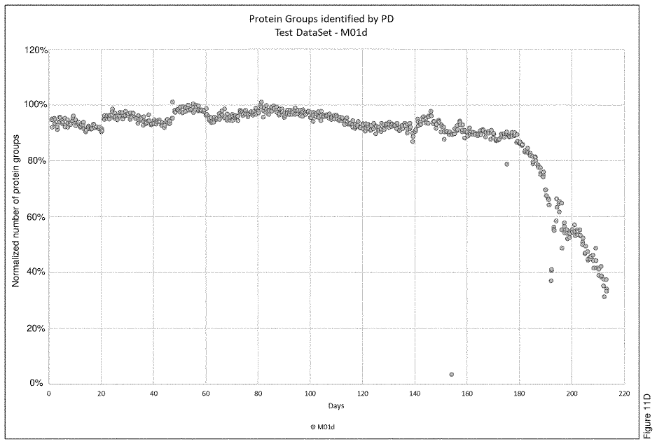

Details about the length of each sequence and the corresponding number of protein groups identified by the Proteome Discover are illustrated in FIGS. 11A to 11D. The number of protein groups is normalized to a relative scale. The data (M01a-M01d) were obtained when the four different sequences were run on the same LC-MS setup (M01) using the same LC-MS methods and same MS and LC. M01a, M01b and M01c were used as training datasets, whereas M01d was used as a validation dataset to predict the number of protein groups by machine learning. These predictions are compared to the number of protein groups obtained by processing the validation data with the Proteome Discoverer software.

To demonstrate the effectiveness of the proposed methods that use machine learning to predict the number of protein groups, the number of protein groups identified by Proteome Discoverer is compared to the predicted number of protein groups by the proposed machine learning methods. The machine learning algorithm was trained by supplying 37 parameters (characteristics) extracted from the raw file data (the .raw files). These parameters can be obtained quickly from the raw file data (<5 minutes per raw file) as they represent absolute, relative or median values from the MS1 and MS2 spectra.

Table 1 below provides an overview of the parameters (and variations) used for training the machine learning algorithm.

| | | Grouped | |
|---|---|---|---|
| | MS- | additionally | |
| Parameter | Level | by AGC | Type |
| AGC Fill | 2 | no | Median |
| AGC Fill Rate [AGF | 2 | yes | Relative Value |
| Fill = 1/AGC Fill ALL] | | | |
| Baseline | 2 | no | Median |
| Charge | 2 | no | Median |
| Intensity | 2 | no | Median |
| Ion Injection Time [s] | 1, 2 | no, yes | Median |
| ITMS2 theoretical | 2 | yes | Median |
| Ion Injection Time [s] | 1 | no | Median |
| Noise | 2 | no | Median |
| Number of Spectra | 1, 2 | no | Absolute Value |
| Number Peaks MS1 | 1, 2 | no | Median |
| Precursor m/z [Da] | 1 | yes | Median |
| Precursor S/N | 1 | yes | Median |
| PrOSA Comp (Proportional | 1 | no | Median |
| Orbitrap Signal Adjustment) | | | |
| Ratio MS1/MS2 Spectra | 1&2 | | Relative Value |
| RawOvFtT (and variations) | 2 | yes | Median |
| Res. Dep. Intens | 2 | yes | Median |
| Resolution | 2 | | Median |
| TIC (isolation Window) | 1 | yes | Median |
| Total Ion Current | 1, 2 | yes | Median |
| (and variations) | | | |

Collecting sufficient precursor ions for MS2 fragmentation requires a specific duration, which depends on the intensity of the precursor ion in the MS1 spectrum. When the intensity of the precursor ion is small, the collection duration is long. To limit the time for collecting precursor ions for MS2 fragmentation, a maximum ion injection time (Max-IT) is defined in the LC-MS method. If the actual time for collecting precursor ions is below the Max-IT, the value for “AGC Filter” equals 1. If the actual time for collecting precursor ions reaches the Max-IT, the value for “AGC Filter” is less than 1. Where the parameters above specify that [AGC Filter=1], this characteristic is based on data collected in cases where the actual time for collecting precursor ions is below the Max-IT (data collected in cases where the actual time for collecting precursor ions reaches the Max-IT being excluded).

“RawOvFtT MS2” is an unscaled filling (ion count) of the Orbitrap FT cell.

“Res. Dep. Intens” is Resolution Dependent Intensity, which is the TIC of the high-resolution scan in relation to the TIC of the MS1 prescan.

In the specific example of the first experiment, the characteristic parameters listed above were extracted from the raw data and used to train the machine learning algorithm. Nevertheless, the machine learning algorithm is not limited to using these specific characteristics as inputs. The ML algorithm can be trained and can make a prediction of the sample metric value based on any combination of:
characteristics extracted from the raw file data;
parameters from the LC;
parameters from calibration files; and/or
parameters from another ultra-fast database search engine.

This makes the proposed methods very flexible and adjustable to different needs to evaluate QC runs.

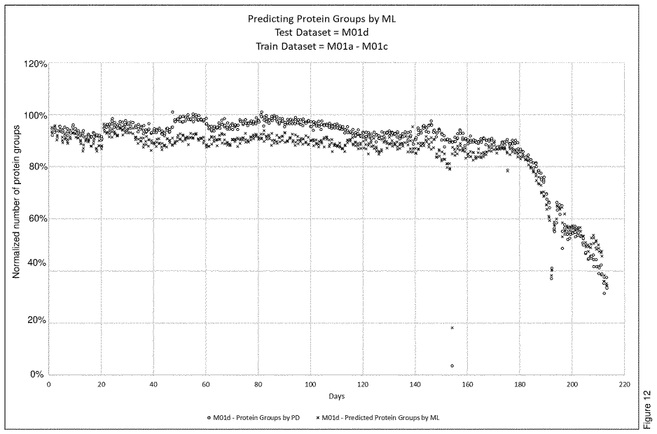

For the validation dataset M01d, 638 separate raw files were obtained (one raw file for each QC run), which represents 213 days of LC-MS measurements. For all measurements, the number of protein groups are obtained by analysing the raw file using the Proteome Discoverer software. These results are used to demonstrate the feasibility of applying machine learning to predict the number of protein groups using only the characteristics extracted from the raw data. FIG. 12 illustrates, for each QC run in the validation dataset, the number of protein groups predicted by extracting the characteristics from the raw data file and providing these characteristics to the ML algorithm, as well as the number of protein groups determined by supplying the raw data file to the Proteome Discoverer software. The number of protein groups is normalized to a relative scale.

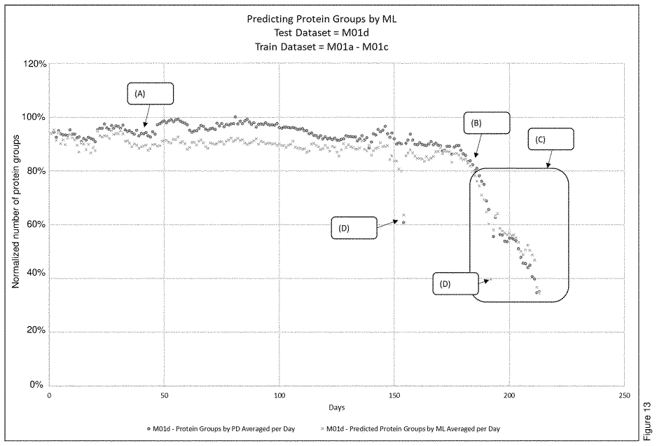

To simplify the data presentation, FIG. 13 illustrates the average number of protein groups for each day, rather than the number of protein groups determined for each QC run. FIG. 13 therefore shows, for each day during which data were collected in the validation dataset, the average number of protein groups predicted by machine learning, compared to the average number of protein groups obtained by the Proteome Discoverer software. The number of protein groups is normalized to a relative scale.

As can be seen in FIG. 13, the results demonstrate that a sample metric value may be predicted effectively over a complete sequence (the M01d validation dataset) using machine learning and only providing raw file related characteristics. The predict sample metric value (number of protein groups) had an absolute error of 117 protein groups.

Importantly, the performance decrease of the LC-MS setup observed towards the end of the sequence is accurately predicted by the ML algorithm. Therefore, the sample metric value predict by the ML algorithm may be reliably used to determine a QC outcome for the LC-MS.

Other specific points/areas are labelled in FIG. 13, which are discussed more detail below.

Predicting the protein groups for the first 50 days was fairly precise, with an average discrepancy of −3%.

At day 47 (Label (A) in FIG. 13), the HeLa sample was replaced, which has a direct influence on the prediction of protein groups by machine learning (average change of −6% for day 50-day 150).

With increasing run-time of the LC-MS setup, the performance of the LC-MS setup starts to be impacted negatively at day 180 (Label (B) in FIG. 13). From this day on, the protein groups decrease further from a value of approx. 2200 to 800 protein groups at the end of the sequence at day 213.

This negative impact on the LC-MS performance can also be determined by the machine learning algorithm. This is highlighted with box (C) in FIG. 13.

Some clear outliers/drop-out of the LC-MS performance are observed at day 154 and day 192. These are predicted precisely by the ML algorithm, as there is strong agreement with the PD software (Label (D) in FIG. 13).

In a second experiment, data was obtained during a sequence using the second OE480 LC-MS instrument setup (M02). The dataset is referred to as “M02d2”. The data from this sequence was used to test how well a machine learning algorithm trained using data from a first LC-MS setup M01 can predict the number of protein groups for a different LC-MS setup M02. Additionally, this sequence was running for 257 days, which is approximately 40 days longer than the final sequence shown in the first experiment (M01d).

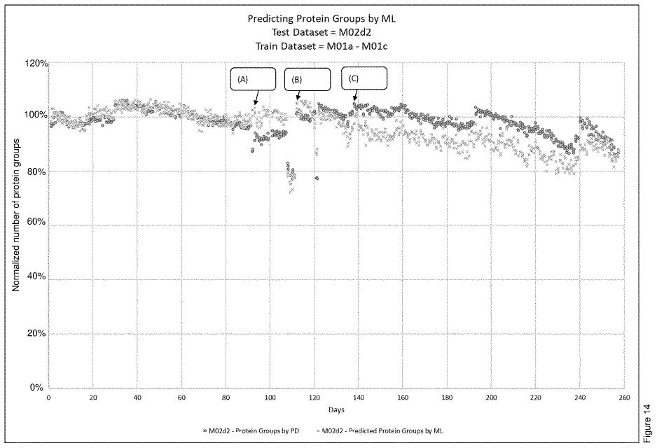

Overall, the mean absolute error for predicting the entire sequence M02d2 was 126 protein groups. FIG. 14 illustrates the predicted number of protein groups by machine learning and the number of protein groups determined by Proteome Discoverer. Here, as with FIG. 13, multiple results from a single day are averaged and shown as a single data point for a single day. The number of protein groups is normalized to a relative scale.

The prediction was much better for approximately the first 90 days. Starting with day 92 (labelled with (A) in FIG. 14), the LC connected to the MS showed pressure instabilities which required manual intervention (including a column exchange). This new column only lasted a few days. Another exchange of the column was needed at day 112 (labelled as (B) in FIG. 14). During the time frame from day 112 to day 134, the predictions made by ML matched the data obtained from PD well. Starting with day 134 (labelled as (C) in FIG. 14), the instrument was relocated into another lab, which had a direct effect on the number of protein groups identified (the average change is about-8%).

For the third LC-MS instrument setup (M03), data from only one sequence was recorded. As with the first and second experiments, the machine learning algorithm was trained using data obtained during the first three sequences run on the first instrument (M01a-M01c). The machine learning algorithm was then used to predict the sample metric value for the third instrument (M03).

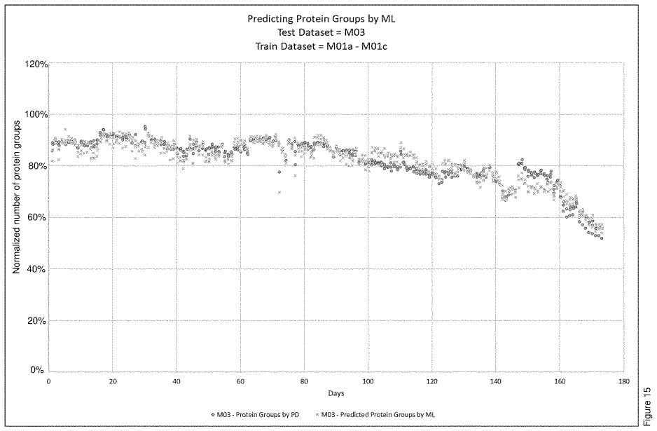

FIG. 15 illustrates prediction of the number of protein groups by machine learning compared to the number of protein groups obtained by Proteome Discoverer. Here, results from a single day are merged and shown as a single data point for a single day. The number of protein groups is normalized to a relative scale.

Overall, the prediction from the ML algorithm was very precise with a mean absolute error of 60 protein groups. This low deviation between ML predicted and Proteome Discoverer results is seen for nearly all days. Notably, there is a small deviation at approximately day 150.

In a fourth experiment, the number of protein groups for the M01d validation data set was predicted by a ML algorithm trained using the M01a-M01c data sets, assisted by results from a fast database search engine.

As described above, QC runs can also be evaluated using Search Engines, which are designed to be ultra-fast. One of these ultra-fast Search Engines, “Morpheus”, was also used to analyse the raw files from the M01d validation data set. Morpheus reports the number of protein groups, Peptides and PSMs (among others). These three parameters were used to train the machine learning algorithm in two different ways:
a) Using only results obtained by Morpheus and
b) Using results obtained by Morpheus, along with characteristics extracted from the raw file.

Using Morpheus search results (alone or in combination with characteristics extracted from the raw file) as input parameters to the ML algorithm was tested with the validation dataset M01d from the first experiment. In both training methods, the mean absolute error was very low. This demonstrates generally that an external ultra-fast search engine can also be used to predict the number of protein groups by machine learning, without running a database search with Proteome Discoverer. The mean absolute error was:
a) Only Morpheus: 71 protein groups
b) Morpheus+raw file related characteristics: 61 protein groups

This is an improvement of 40 protein groups when comparing to the first experiment, where only raw file related parameters were used (the mean absolute error from the first experiment was 117 protein groups).

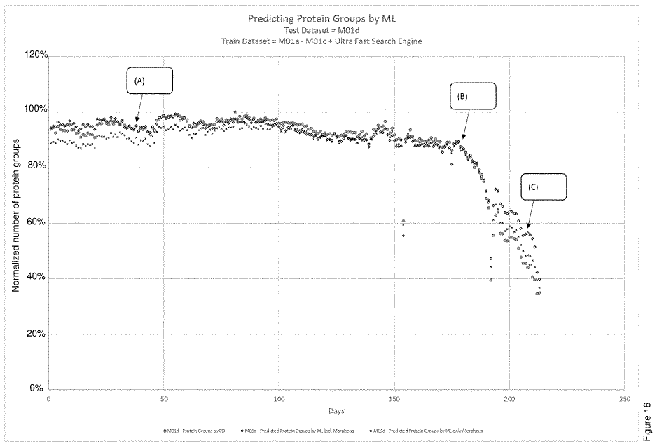

The number of protein groups for the M01d validation data set are shown in FIG. 16. In this data, results from a single day are merged and shown as a single data point for each day. The number of protein groups is normalized to a relative scale. In the graph illustrated in FIG. 16, the predicted number of protein groups from the machine learning algorithm is compared with the number of protein groups obtained by Proteome Discoverer.

Accordingly, there are three data series illustrated in the graph in FIG. 16:
A) the number of protein groups obtained by Proteome Discoverer
B) the number of protein groups predicted by an ML algorithm trained using only Morpheus Results
C) the number of protein groups predicted by an ML algorithm trained using Morpheus Results combined with raw file related characteristics

Where the ML algorithm was trained using only identification results from the ultra-fast database search (Morpheus), the training dataset for the ML algorithm contained only the number of protein groups, unique peptides and PSMs from Morpheus.

As the ML algorithm was only trained with the results from the ultra-fast database search, the input for prediction the protein groups from PD is also based on the results of the ultra-fast database search engine. As a result, the parameters from the ultra-fast search engine are used to predict the number of protein groups (and compared to the number of protein groups reported by Proteome Discoverer). In this example, no plurality of characteristics extracted from the raw file are used as inputs to the ML algorithm (either in training or prediction).

Overall, the prediction was slightly better than using only characteristics extracted from the raw file. This is not surprising as both search engines pre-process MS2 spectra to map theoretical peptides to this spectrum. Further, three identification results from Morpheus are used to predict one result from Proteome Discoverer (the number of protein groups).

As outlined above, Morpheus and Proteome Discoverer are both search engines which map MS2 spectra to peptides. The characteristics extracted from the raw file are different to this. They are absolute or median values of very specific and selected parameters that are expected to change only a very minor amount when the LC-MS setup performs as expected. As a result, training the machine learning algorithm with both kinds of data is advantageous:
results from another search engine (Morpheus), which is data of the same kind as the output sample metric value; and
characteristics extracted from the raw file, which contains orthogonal data.

In this example, the results from the ultra-fast database search are combined with the characteristics extracted from the raw file to train the machine learning algorithm and as inputs to the machine learning algorithm for the prediction.

The ultra-fast database search engine performs similar tasks to the Proteome Discoverer software. However, Proteome Discoverer uses much more sophisticated approaches to identify proteins in raw file data than the ultra-fast database search does. As these sophisticated approaches need more computing time, the overall processing time of a raw file by Proteome Discoverer is longer but it contains more “useful” information for the user, such as quantification values.

Generally, the value of the sample metric provided by the Proteome Discoverer is considered as the closest to the true value (since this is the most sophisticated means by which the value is derived). Parameters derived via an ultra-fast database search may give a good approximation. However, these parameters may not be sufficiently accurate for the purposes of determining a QC outcome. Notably, the combination of the ultra-fast database search parameters and characteristics extracted from the raw files outperforms the use of an ultra-fast search engine alone.

This combination of parameters to train the machine learning algorithm has shown the best performance experimentally, with an average absolute mean of 61 protein groups.

As can be seen from the two different approaches to training the ML algorithm in the fourth experiment above, various different input parameters can be used to train the ML algorithm. As will be appreciated by the skilled person, various different combinations or sub combinations of the characteristics and parameters mentioned in this application may be used as inputs to the ML algorithm.

This is true for the first 50 QC runs, which were difficult to predict using only Morpheus related parameters for the Training Dataset (see FIG. 16, Label (A)).

The time range from day 50-150 was difficult to predict in the first experiment, due to the change of the HeLa samples. By combining Morpheus and the raw file related parameters, and using this combination to train the ML algorithm, the agreement between the predicted sample metric value from the ML algorithm and the sample metric value obtained via Protein Discoverer is very good.

For evaluating QC runs, it is important to monitor if (or when) a LC-MS system is impacted. This negative impact starts with day 180 for M01d where the number of protein groups identified tends to decrease. This decrease can also be observed in the predicted number of protein groups (illustrated with Label (B) in FIG. 16). Towards the end of the sequence (around day 190) when the LC-MS setup was significantly impacted by lower numbers of identifiable protein groups, (1500 or fewer, compared to a starting value of 2200), the values predicted by a ML algorithm trained with a combination of raw file related characteristics and the ultra-fast Search Engine identifications appears slightly less precise.

In some further examples, the predictions of the number of protein groups from the machine learning algorithm are combined with (or compared to) results from an ultra-fast database search. This will increase the level of certainty regarding the prediction of the data. The prediction can also be accompanied by classical QC data evaluation processes, such as the effective gradient length, FWHM and retention time stability of spiked in peptides (e.g., Thermo PRTC) or the sensitivity (e.g., Thermo System Suitability Standard). All these evaluation results could be passed into a final evaluation.

Where this document refers to determining or predicting “a number of protein groups”, this is equivalent to determining or predicting the “Total Number of Protein Groups (identified)” parameter from the Proteome Discoverer software.

The unit Dalton (Da) is used in this description as an alternate name for the unified atomic mass unit. Hydrogen has a mass of 1 Da. A Hydrogen ion is seen at m/z=1. The m/z ratio of Hydrogen is 1.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that, for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise.

Unless otherwise defined, all other technical and scientific terms used herein have the meaning commonly understood by one of ordinary skill in the art to which this invention belongs. In case of conflict, the present specification, including definitions, will control. It will be appreciated that there is an implied “about” prior to the quantitative terms mentioned in the present description, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. As used herein, “a” or “an” also may refer to “at least one” or “one or more.” Also, the use of “or” is inclusive, such that the phrase “A or B” is true when “A” is true, “B” is true, or both “A” and “B” are true.

As used in this document, the term “scan”, when used as a noun, means a mass spectrum, regardless of the type of mass analyzer used to generate and acquire the mass spectrum. When used as a verb herein, the term “scan” refers to the generation and acquisition of a mass spectrum by a method of mass analysis, regardless of the type of mass analyzer or mass analysis used to generate and acquire the mass spectrum. As used herein, the term “full scan” refers to a mass spectrum than encompasses a range of mass-to-charge (m/z) values that includes a plurality of mass spectral peaks.

As used in this document, each of the terms “liquid chromatograph” and “liquid chromatography” (both abbreviated “LC”) as well as the term “Liquid Chromatography Mass Spectrometry” (abbreviated as either “LCMS” or “LC-MS”) are intended to apply to any type of liquid separation system that is capable of separating a multi-analyte-bearing liquid sample into various “fractions” or “separates”, where the chemical composition of each such “fraction” or “separate” is different from the chemical composition of every other such fraction or separate, wherein the term “chemical composition” refers to the numbers, concentrations, and/or identities of the various analytes in a fraction or separate. As such, the terms “liquid chromatograph”, “liquid chromatography” “Liquid Chromatography Mass Spectrometry”, “LC”, “LCMS” and “LC-MS” are intended to include and to refer to, without limitation, liquid chromatographs, high-performance liquid chromatographs, ultra-high-performance liquid chromatographs, size-exclusion chromatographs and capillary electrophoresis devices.

Instead of the LC device any other separation device, including an ion mobility device, HPLC, GC or ion chromatography could be interfaced to the mass spectrometer. Also any known fragmentation method (including collisionally activated dissociation, photon induced dissociation, electron capture or electron transfer dissociation) produces data suitable for use with the invention.

1. A method for predicting one or more sample metric values by machine learning, the method comprising:
determining a plurality of characteristics from raw data obtained by analysing a proteomics sample in a liquid chromatography mass spectrometer, LC-MS; and
predicting one or more sample metric values by processing the plurality of characteristics using one or more trained machine learning models.

2. The method of claim 1, wherein the machine learning model is trained using data obtained from a plurality of LC-MS combinations and/or from one or more LC-MS instruments exhibiting a plurality of different LC-MS performance states.

3. The method of claim 1, wherein a data size of the plurality of characteristics is at least 10 times smaller than a data size of the raw data, preferably 100 times smaller, more preferably 1000 times smaller.

4. The method of claim 1, wherein the machine learning algorithm only processes the plurality of characteristics and does not directly process the raw data.

5. The method of claim 1, wherein a time taken to determine the plurality of characteristics from the raw data is less than a time taken to obtain the raw data by analysing the proteomics sample, preferably at least ten times less.

6. The method of claim 1, wherein analysing the proteomics sample in the LC-MS comprises performing an LC-MS run, wherein the raw data comprises data relating to a retention time during the LC-MS run, the method further comprising:
dividing the retention time into a plurality of retention time slices; and
manipulating the raw data to provide data relating to each of the plurality of retention time slices,
wherein the plurality of characteristics comprises a plurality of characteristics for each retention time slice.

7. A method of training a machine learning model for predicting a sample metric value, the method comprising:
determining a plurality of characteristics from raw training data obtained by analysing a training sample in a LC-MS;
processing the raw training data via a database search to determine the sample metric value for the training sample; and
training the machine learning model using the plurality of characteristics and the sample metric value for the training sample determined via the database search, so that the machine learning model is trained to predict the sample metric value for a subsequent proteomics sample, based on a plurality of characteristics determined from raw data obtained by analysing the subsequent proteomics sample in a LC-MS.

8. The method of claim 7, further comprising training the machine learning model using one or more of:
features from the LC,
features from one or more calibration files and/or
features from another database search engine.

9. The method of claim 7, further comprising validating the machine learning model by:
determining a plurality of characteristics from raw validation data obtained by analysing a validation sample in a LC-MS;
predicting a sample metric value for the validation sample by processing the plurality of characteristics using the machine learning model;
processing the raw validation data via a database search to determine the sample metric value for the validation sample; and
comparing the sample metric value determined by processing the raw validation data via the database search with the sample metric value predicted by processing the plurality of characteristics using the machine learning model.

10. A method of determining a data quality for raw data obtained by analysing a proteomics sample in a liquid chromatography mass spectrometer, LC-MS, the method comprising:
predicting one or more sample metric values using the method of claim 1;
based on the one or more sample metric values, determining whether a data quality for the raw data is above a threshold data quality;
if the data quality for the raw data is above a threshold data quality, performing further data analysis on the raw data; and
if the data quality for the raw data is not above the threshold data quality, skipping further data analysis for the raw data.

11. A method of determining a quality control, QC, outcome for a liquid chromatography mass spectrometry, LC-MS, process, the method comprising:
in a QC run, predicting one or more sample metric values by analysing a proteomics sample in a liquid chromatography mass spectrometer, LC-MS, using the method of claim 1, wherein the proteomics sample is a quality control, QC, sample, wherein the one or more sample metric values comprises a QC sample metric value; and
based on the QC sample metric value, determining whether the QC outcome is a pass or a fail.

12. A method of performing a liquid chromatography mass spectrometry sequence, the sequence comprising one or more cycles, each cycle comprising the following steps:
performing a quality control, QC, run and determining a QC outcome via the method of claim 11,
if the QC outcome is a fail, ending the sequence without further cycles; and
if the QC outcome is a pass, performing one or more sample analysis runs and proceeding to the next cycle.

13. A liquid chromatography mass spectrometer, LC-MS, configured to perform the method of claim 1.

14. Computer software comprising instructions that, when executed by a processor of a computer, cause the computer to perform the method of claim 1.
